(19) United States
(12) Patent Application Publication
(10) Pub. No.: US 2025/0093327 A1
(43) Pub. Date: Mar. 20, 2025

(54) STOCHASTIC HEATING AT AN ELECTROCHEMICAL INTERFACE

(71) Applicants: Kansas State University Research Foundation, Manhattan, KS (US); Lawrence Livermore National Security, LLC, Livermore, CA (US)

(72) Inventors: Bret N. Flanders, Manhattan, KS (US); Krishna R. Panta, Manhattan, KS (US); Christine Orme, Livermore, CA (US)

(21) Appl. No.: 18/729,592

(22) PCT Filed: Jan. 17, 2023
(86) PCT No.: PCT/US2023/060745

§ 371 Date: Jul. 17, 2024

Related U.S. Application Data
(60) Provisional application No. 63/300,110, filed on Jan. 17, 2022.

(51) Int. Cl. G01N 33/483; G01N 27/416; G21B 1/03

(52) U.S. Cl. CPC G01N 33/4833; G01N 27/416; G21B 1/03

(57) ABSTRACT

Methods and apparatus for stochastically heating charged reactants by applying a random (stochastic) voltage signal to a working electrode, thereby inducing a stochastic electric field. By agitating the charged species in the interfacial region adjacent the working electrode or other target substrate, the stochastic electric field increases the effective temperature of the charged species while scarcely affecting any surrounding neutral molecules (e.g., water). This effect increases the reaction rates in the interfacial region and can allow the reactants to achieve rates that are commensurate with physically inaccessible temperatures in common solutions.

### CROSS-REFERENCE TO RELATED APPLICATIONS

The present application claims the priority benefit of U.S. Provisional Patent Application Ser. No. 63/300,110, filed Jan. 17, 2022, entitled STOCHASTIC HEATING AT AN ELECTROCHEMICAL INTERFACE, incorporated by reference in its entirety herein.

### STATEMENT REGARDING FEDERALLY SPONSORED RESEARCH OR DEVELOPMENT

This invention was made with government support under Contract No.: EPSCoR-1430519 awarded by the National Science Foundation and Contract No.: 1 R21EY026392 awarded by the National Institute of Health. The government has certain rights in the invention.

### BACKGROUND OF THE INVENTION

### Field of the Invention

The present invention relates to methods for amplifying reaction rates of charged (ionic) reactants using stochastic heating.

### Description of Related Art

Diffusion-limited chemical rate constants k are characterized by the Arrhenius rate law:

$$k ∝ e = \frac{Barrier Height}{Fluctuation Strength} {e}^{- \frac{{E}_{a}}{{k}_{B} T}} \qquad ( 1 )$$

where Eₐ is the activation energy, k_{B} is Boltzmann's constant, and T is the temperature. Conceptually, this relationship says that thermal fluctuations provide the forces that drive a reactant to the product state, and do so with a probability that diminishes as the energetic barrier increases. This picture suggests two approaches to controlling chemical reaction rates: 1) reducing the barrier height or 2) intensifying the fluctuation strength. The relevant literature for this invention revolves around these two approaches.

The field of electrochemistry is based on the first approach: using voltages to vary the activation energy of electrode-enhanced reactions, thereby controlling their rates. A similar feat may be done with biological cells. For example, it is an experimental fact that external electric fields affect the transport rates of membrane bound-enzymes in biological cells. Previous studies have investigated the effect of weak electric fields on biological cells. Because external electric fields are intensified in and near the membranes of biological cells, weak fields can amplify the activity of membrane-bound enzymes that have charged moieties. Others demonstrated this effect by showing that enzymes responsible for ferrying substrates into a cell speed up by capturing energy from an external electric field. Hence, this process is essentially an electrochemical reaction: the applied field activates a chemical step by reducing the barrier height for that step.

While the second approach noted above is often as prosaic as turning up the temperature on a flask of chemicals, the counter-intuitive concept of mechanistically beneficial noise emerged as a new way to modulate the fluctuation factor in the Arrhenius equation when others conceived of stochastic resonance as a possible mechanism for periodic, solar-induced ice-ages. The basic idea of stochastic resonance is that external noise intensifies the strength of the fluctuations that the system undergoes, thereby tuning its Arrhenius rate k into resonance with a weak, periodic external force.

### SUMMARY OF THE INVENTION

The present invention is broadly concerned with stochastic heating, a special case of stochastic resonance where there is no periodic external force. In a clear demonstration of stochastic heating, one study observed the instantaneous position of a charged polystyrene micro-sphere in an optical double well potential while applying electrical white-noise to the system. The noise exponentially amplified the well-to-well transition rate of the sphere, just as if its Arrhenius rate had been elevated by increasing the temperature of the system. This works because, fundamentally, the temperature of a particle reflects its mean kinetic energy. Hence, the electrical noise, which elevates a charged particle's average kinetic energy, increases the fraction of particles with energy sufficient to cross the energy barrier and, thereby, amplifies its transition rate (See FIG. 1). The agitated charged species in solution will have an effective (kinetic) temperature T_{Eff} that is greater than the actual temperature of the surrounding bath T:

$${T}_{E f f} = T + k {Γ}^{2} , \qquad ( 2 )$$

where Γ is the amplitude of the noise, and k is a constant that converts the noise intensity to temperature.

In particular, the present invention is directed to a method for increasing the effective temperature of charged reactants and, thereby, amplifying the reaction rates that they exhibit. This effect occurs by applying a stochastic (i.e., noisy) voltage signal to a working electrode (in an electrochemical cell) where the charged reactants at the working electrode or other target substrate are oxidized or reduced. The intensified electric field near the working electrode or other target substrate randomly agitates the charged species in the high field region, causing them to mimic the kinetic activity they would have were the bath temperature elevated (see FIG. 2). However, because most other molecules in the solution (e.g., water molecules) are uncharged, they are largely unaffected by the field. In this sense, the field selectively heats the charged reactants. In essence, the stochastic signal catalyzes the reactions that the charged species undergo, permitting reaction rates commensurate with physically inaccessible temperatures (i.e., beyond the solvent-boiling point) to be realized.

The prevailing approach towards controlling electrochemical reactions is to minimize the levels of external noise sources. The present invention does the opposite, and it is this difference that sets the present invention apart from prior electrochemical efforts.

Methods according to embodiments described herein can stochastically heat charged reactants in solution by applying a random voltage signal to a working electrode in an electrochemical cell where the reactants are oxidized or reduced. By agitating the charged species in the interfacial region, the stochastic field increases their effective temperature while scarcely affecting surrounding neutral molecules (e.g., water). This effect causes the reactants to achieve rates that are commensurate with physically inaccessible temperatures.

There are several key features that can be adapted to promote reactions. First, ions near interfaces interact with very large electric fields that increase their kinetic energy similar to an increase in temperature. However, in contrast to heating, electric fields only couple to the ions, not the neutral molecules. Thus, ions can reach effective temperatures above the boiling point of water for solutions that remain at room temperature. Second, the interface can either be an electrode surface or an unwired (floating) surface that is responding with image charges. Third, conductive spheres (like cells or vesicles or droplets) further increase the electric field due to their geometry.

In one embodiment, there is provided a method of amplifying a reaction rate of one or more ionic reactants within a reaction system comprising the one or more ionic reactants and at least one non-ionic material. The method comprises: a) generating a stochastic signal with electronic signal generation equipment; and b) transmitting the stochastic signal to an electrode, the electrode applying the stochastic signal to the reaction system and inducing a stochastic electric field within the reaction system. The induced stochastic electric field operates to increase the kinetic energy of the one or more ionic reactants to a greater extent than the kinetic energy of the at least one non-ionic material is increased.

In another embodiment, there is provided a method of amplifying an electrodeposition reaction rate of one or more ionic metal reactants within a reaction system comprising the one or more ionic metal reactants dispersed or dissolved within a solvent. The method comprises applying a stochastic electric signal to the reaction system, thereby inducing a stochastic electric field within at least a portion of the reaction system. The induced stochastic electric field operates to increase the kinetic energy of at least a portion of the one or more ionic metal reactants to above the kinetic energy of the one or more ionic metal reactants at the boiling point of the solvent.

In another embodiment, there is provided a method of amplifying a reaction rate of one or more ionic reactants within a reaction system comprising the one or more ionic reactants dissolved within a solvent and having a system temperature. The method comprises applying a stochastic electric signal to the reaction system, thereby inducing a stochastic electric field within at least a portion of the reaction system and increasing the kinetic energy of at least a portion of the one or more ionic reactants without increasing the system temperature above the boiling point of the solvent. The portion of the one or more ionic reactants has increased kinetic energy reacts to form a precipitate product.

In another embodiment, there is provided a method of stochastically heating a DNA molecule in a lipid vesicle dispersed within a reaction system. The method comprises: applying a first electric signal to the reaction system, thereby inducing an electric field adjacent a membrane of the lipid vesicle and trapping the DNA molecule within a screening layer formed by the electric field; and applying a stochastic electric signal to the reaction system, thereby increasing the kinetic energy of the trapped DNA molecule.

In another embodiment, there is provided apparatus for amplifying a reaction rate of one or more ionic reactants within a reaction system comprising the one or more ionic reactants and at least one non-ionic material. The apparatus comprises: electronic signal generation equipment configured to generate a stochastic signal; a working electrode in electronic communication with the electronic signal generation equipment and disposed within the reaction system, the working electrode configured to apply the stochastic signal to the reaction system and induce a stochastic electric field within the reaction system. The induced stochastic electric field operates to increase the kinetic energy of the one or more ionic reactants to a greater extent than the kinetic energy of the at least one non-ionic material is increased.

### BRIEF DESCRIPTION OF THE DRAWINGS

FIG. 1 is a schematic graph showing the increase of kinetic energy due to the stochastic electric field, according to embodiments of the present invention;

FIG. 2 is a schematic illustration of the charged ionic reactants subjected to increased kinetic energy adjacent the target substrate, according to embodiments of the present invention;

FIG. 3A is a graph showing the electric field strength relative to the distance from the electrodes, according to embodiments of the present invention;

FIG. 3B is a graph showing the electric field strength from a stochastic (noisy) signal relative to the distance from the electrodes, according to embodiments of the present invention;

FIG. 4A is 2D plot of the potential around a vesicle in a 1.0 V·m¹ field, with the equipotential lines decreasing from left to right;

FIG. 4B is a graph showing the field strength required to raise the temperature 1 K, 10 K, and 100 K versus molecular mass with typical values for ions and DNA shown;

FIG. 5A is a schematic of an electrochemical cell used to characterize the AuI→Au0 reaction;

FIG. 5B is a schematic graph showing the potential energy curve for the reaction depicted in FIG. 5A, with the graph minima aligned with the illustrations of the reactant (upper panel) and product (lower panel) states;

FIG. 6A is a graph showing Arrhenius behavior of the AuI→Au0 reaction rate (expressed as j) as the temperature is raised to 376 K;

FIG. 6B is a graph showing the reaction rate as the noise-amplitude applied to the cell is increased (filled circles), with a thermal data line overlying the reaction rate data;

FIG. 7 is a graph showing the silver deposition rate with and without stochastic noise was repeatedly measured over a period of 250 seconds;

FIG. 8 is a graph showing the effective temperature at different field strengths for the silver deposition system;

FIG. 9A is a photograph showing silver deposition on the electrode applying stochastic noise;

FIG. 9B is a photograph showing no silver deposition on the electrode without stochastic noise;

FIG. 10 is a graph showing calcium carbonate growth rate with and without stochastic noise;

FIG. 11 is a graph showing calcium carbonate areal coverage with and without stochastic noise;

FIG. 12A is a photograph showing calcium carbonate nucleation density with stochastic noise;

FIG. 12B is a photograph showing calcium carbonate nucleation density without stochastic noise;

FIG. 13 is a graph comparing calcium carbonate growth rates with and without stochastic noise;

FIG. 14A is a pair of graphs showing calcium carbonate nucleation rates and size with and without stochastic noise;

FIG. 14B is a pair of graphs showing calcium carbonate growth rates and size with and without stochastic noise;

FIG. 15A is a graph showing growth rates of magnesium carbonate at different stochastic signal voltages;

FIG. 15B is a graph showing normalized growth rates over time of magnesium carbonate at different stochastic signal voltages;

FIG. 15C is a graph showing normalized growth rates of magnesium carbonate at different stochastic signal voltages;

FIG. 16A is a photograph showing magnesium carbonate deposition after 7000 seconds at 3.5 V stochastic signal;

FIG. 16B is a photograph showing magnesium carbonate deposition after 7000 seconds at 0.5 V stochastic signal;

FIG. 17 is a graph showing the areal coverage of magnesium carbonate deposition after 2 hours at different stochastic signal voltages;

FIG. 18 is a set of photographs showing the magnesium carbonate structures formed after 2 hours with no noise, 2.5 V, and 3.5 V;

FIG. 19 is a set of photographs showing the progression of nucleation at Vᵣₘₛ=1.0 V over time;

FIG. 20 is a set of graphs and images showing the copper sulfide growth rate and total mass deposited over time using 600 mV stochastic signal (top) and no noise (bottom);

FIG. 21A is a photograph of zinc oxide deposition after 2 days with stochastic noise;

FIG. 21B is a photograph of zinc oxide deposition after 2 days with no stochastic noise;

FIG. 22 is a graph showing the zinc oxide growth rate and total mass deposited over time with and without stochastic noise;

FIG. 23 is a schematic diagram of a stochastic heating system having an unwired “floating” electrode, in accordance with embodiments of the present invention;

FIG. 24 is a graph showing the bandwidth of the stochastic signal applied to a calcite growth using the unwired electrode system shown in FIG. 23;

FIG. 25A is a photograph showing calcite deposition after 12 hours on the unwired electrode using 1.0 Vrms stochastic signal;

FIG. 25B is a photograph showing calcite deposition after 12 hours on the unwired electrode using 0.7 Vrms stochastic signal;

FIG. 25C is a photograph showing calcite deposition after 12 hours on the unwired electrode using 0.3 Vrms stochastic signal;

FIG. 25D is a photograph showing calcite deposition after 12 hours on the unwired electrode using 0 Vrms stochastic signal;

FIG. 26 is a graph showing the Raman analysis confirming calcite growth on the unwired electrode;

FIG. 27 is a graph showing the measured the system temperature versus time over a ˜2 hour period of 1.0 Vᵣₘₛ stochastic excitation for calcite growth on the unwired electrode;

FIG. 28 is a schematic illustration of a dsDNA molecule in screening layer near positive electrode in a DC field (left), and the melted, ssDNA following the addition of the stochastic signal (right);

FIG. 29A is a schematic illustration of an electrolyte-containing vesicle in an external field showing the electric potential V;

FIG. 29B is an enlarged schematic view of the membrane region of the left side of the vesicle shown in FIG. 29A, and particularly showing that the slope of the potential is much steeper in this region, and that the amplified field attracts the negative DNA molecule to the membrane (noise effects are also shown);

FIG. 30A is a schematic diagram of a system for stochastically heating at a “floating” vesicle;

FIG. 30B is a circuit diagram of the system of FIG. 30A;

FIG. 30C is a 2D plot of the potential around a vesicle in a field of 1.0 V m−1 along the horizontal axis;

FIG. 30D is an enlarged view of FIG. 30C showing the vesicle membrane interface;

(d) Enlarged view.

FIG. 31 is a schematic graph showing the screening of potential inside a vesicle due to the external, E₀-induced re-distribution of charges in the vesicle (the sharply screened intra-vesicular potential recalls the steep potential profiles near the electrodes in electrochemical cells);

FIG. 32A is a graph showing the cyclic voltammogram of 5 mM AgNO₃ solution with a supporting electrolyte content of 200 mM KNO₃;

FIG. 32B is an expanded view of the exponential deposition region of FIG. 32A, where the example focuses (the dashed line marks the −12.5 mV overpotential applied in this example);

FIG. 33 is a graph showing the spectrum of stochastic signal (the dashed lines mark the sinusoidal frequencies investigated in this example);

FIG. 34A is a graph showing the difference-current profiles collected while applying a −12.5 mV overpotential in the presence and absence of a stochastic signal (the scan rate (in all cases) was 1.8 mV s⁻¹);

FIG. 34B is a graph showing the deposited mass during the voltage sweep measurements shown in FIG. 34A;

FIG. 34C is an optical image of the working electrode after the stochastic voltage sweep (scale bar=3 mm);

FIG. 35A is a graph showing the difference-current density-profiles for the stochastic, 100 kHz sinusoidal, 10 kHz sinusoidal, and DC voltage sweeps FIG. 35B is a graph showing deposited mass profiles recorded during the voltage sweeps in FIG. 35A;

FIG. 35C is an optical image of working electrode after the 10 kHz voltage sweep (scale bar=3 mm);

FIG. 36A is a schematic illustration of apparatus for observing localized stochastic heating at an interface by characterizing 2D Langmuir films, according to embodiments of the present invention; and

FIG. 36B is a graph showing ideal gas behavior of pressure-area isotherms of a Langmuir film.

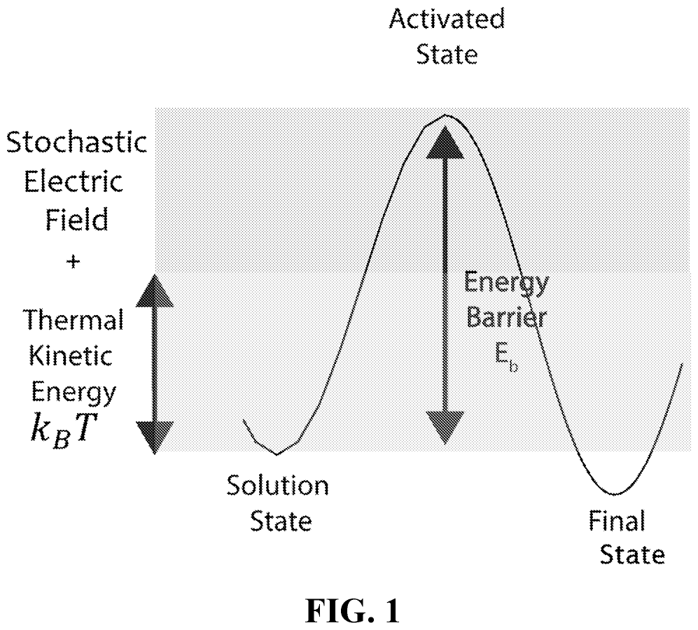

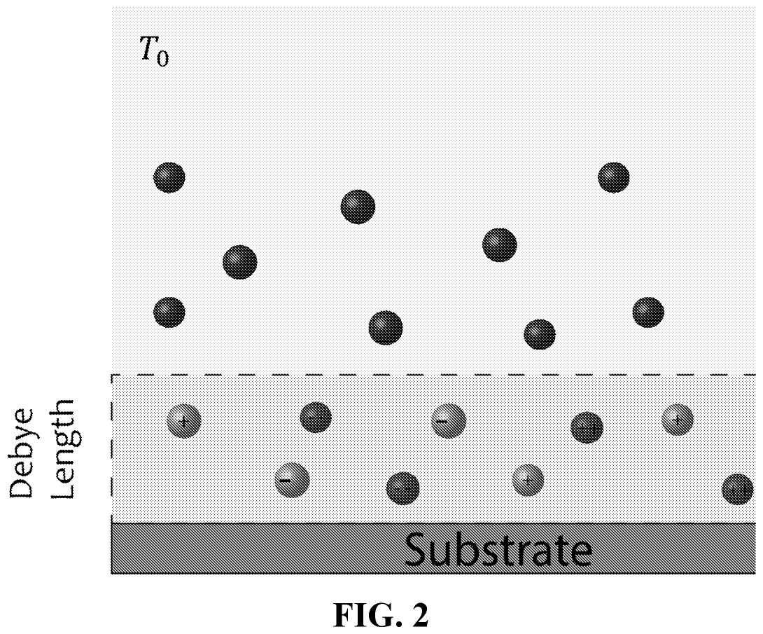

### DETAILED DESCRIPTION

Embodiments of the present invention are generally directed to stochastic heating methods for amplifying (e.g., increasing) a reaction rate of one or more ionic reactants within a reaction system. Advantageously, the stochastic heating methods described herein can increase reaction kinetics without the use of catalysts, enzymes, or elevated reaction system temperatures, although the methods can also be used in conjunction with catalysts, enzymes, and/or elevated temperatures. Surprisingly, it has been discovered that stochastic electric signals impart a greater amplification of reaction rates as compared to constant or sinusoidal signals.

The reaction system generally comprises one or more ionic reactants. In certain embodiments, the reaction system further comprises at least one non-ionic material. In certain embodiments, the one or more ionic reactants are dispersed or dissolved within the at least one non-ionic material. In certain embodiments, the non-ionic material comprises water. Thus, in certain embodiments, the reaction system comprises the one or more ionic reactants dispersed or dissolved within an aqueous solution. However, in certain embodiments, other non-ionic organic and inorganic solvents may also be used. Regardless the solvent used, when present in the reaction system, the one or more ionic reactants may be generally dispersed or dissolved within the non-ionic solvent material, along with other electrolytes present in the reaction system. Other non-ionic solvents may include alcohols and hydrocarbon solvents.

In certain other embodiments, the one or more ionic reactants are present within an ionic liquid. In certain such embodiments, the reaction system comprises an ionic liquid and at least one non-ionic material comprising a neutral (non-ionic) gas or liquid, which can form a separate phase from the ionic liquid.

In certain embodiments, the initial temperature of the reaction system is 290 K to 305 K, although higher and lower initial temperatures may also be used, depending on the particular reaction system and application.

A variety of reactants can be used in accordance with embodiments of the present invention, and the particular reactants can be chosen depending on the desired product or application. However, the one or more reactants should generally comprise ionic molecules that are capable of exhibiting an increase in kinetic energy upon exposure to a stochastic electric field. In certain embodiments, the one or more reactants are dissolvable in a non-ionic solvent material. In certain embodiments, the one or more reactants include acids and salts that have been dissolved into their ionic components (i.e., cation and anion) in the reaction system. Such salts may be comprised of a variety of cationic components, such as hydrogen ion(s) (e.g., from acid salts), ammonium, alkali metals (e.g., Na, Li, K, etc.), alkaline earth metals (e.g., Ca, Mg, etc.), transition metals (e.g., Cu, Fe, Ni, Mn, Ti, Cr, Ag, Au, W, etc.), and others. The reactant salts may be comprised of a variety of anionic components, such as acetates, carbonates, chlorides, citrates, fluorides, nitrates, nitrites, oxides, phosphates, phosphites, sulfates, sulfites, and others. The concentration of the one or more reactants in the reaction solution may generally be 0.1 mM to 50 M, although higher and lower concentrations may also be used. The supporting electrolyte concentration (e.g., Na+, K+, and other non-target reactant cations and anions) of the reaction system may generally be 1 mM to 1 M, although higher and lower concentrations may also be used.

It should be understood that other ionic components (including both cationic and anionic components) not expressly listed herein may also be used, with a wide variety of possible reactants, particularly in biological systems. For example, in certain embodiments, the one or more reactants may comprise enzymes having charged moieties and/or negatively-charged DNA molecules, which may be present within lipid vesicles, or bonded to the membranes thereof, in biological reaction systems.

Methods of stochastic heating generally comprise applying a stochastic electric signal to the reaction system, thereby generating an electric field within at least a portion of the reaction system. The stochastic signal may be generated with electronic signal generation equipment and transmitted to a working electrode disposed within the reaction system. The signal generation equipment can include an electric signal generator and/or modulator (e.g., Agilent 2230 function generator, which has a nominal bandwidth of ˜10 MHz). In certain embodiments, the strength of the stochastic signal is 10 mV_{RMS} to 10 V_{R}MS. Higher and lower signal strengths may also be used, depending on the particular application. In certain embodiments, the stochastic signal has a bandwidth of 100 Hz to 10 MHz. In certain embodiments, the stochastic signal may be characterized, for example, as Gaussian-White Noise, Poisson-White Noise (Shot noise), or Gaussian-Red Noise (Brownian motion). In certain embodiments, the stochastic signal may be characterized, for example, as white noise (power spectrum=constant), pink noise (power spectrum=1/f), or red noise (power spectrum=1/f²).

The working electrode applies the stochastic signal to the reaction system, thereby inducing a stochastic electric field within the reaction system. The working electrode can have a variety of shapes (e.g., flat, hemispherical, pointed) and sizes (e.g., a radius of curvature of the foremost tip ranging between 1 nm and infinity, for a flat electrode). The working electrode can comprise a variety of conductive materials. In certain embodiments, the electrode surface comprises a material selected from the group consisting of gold, tungsten, platinum, silver, ruthenium, and conducting polymers (e.g., polyethylene-dioxythiophene).

In certain embodiments, the stochastic signal applied by the working electrode can induce an electric field at the surface (or growing substrate) of the working electrode and/or one or more other electrodes within the reaction system. Such other electrodes may include grounded electrodes and/or “floating” unwired electrodes. These other electrodes may have surfaces comprising the same material as the working electrode or different materials than the working electrode.

In certain embodiments, the surface energy of the working electrode or other electrodes may be modified, for example, to favor nucleation reactions. Such modification may include, for example, a hydrophobic coating. Exemplary coating materials include copolymers of chlorotrifluoroethylene (CTFE) and vinylidene fluoride, hexamethyldisilazane (HMDS), and silanes.

In certain embodiments, the reaction system comprises one or more lipid vesicles dispersed therein, and the lipid membrane of the vesicle(s) may act similar to the floating unwired electrode described above. In certain such embodiments, the presence of the membrane in electrolyte-containing vesicles can amplify the electric field near the membrane, thereby focusing the potential drop across the vesicle membrane.

In certain embodiments, where the reactants are contained in ionic liquids, the electrode(s) may be positioned at the interface of the ionic liquid and a separate neutral phase. In certain such embodiments, the reaction rate of the one or more reactants may be amplified at the interface so as to produce a desired reaction product (e.g., a film) at the interface, which can be easily removed from the ionic liquid.

The stochastic electric field induced by the working electrode operates to increase the kinetic energy of the one or more ionic reactants within at least a portion of the reaction system, thereby amplifying (i.e., increasing) the reaction rate of the one or more ionic reactants. In certain embodiments, the stochastic electric field amplifies the kinetic energy of the one or more ionic reactants to a greater extent than it increases the kinetic energy of the at least one non-ionic material in the reaction system.

For the stochastic electric field to act on the charged (ionic) reactants in the high field region of the reaction system surrounding the electrode, that high field must change fast enough to follow the rapidly varying stochastic signal. This capability depends on a variety of tunable parameters, such as conductivity of the solution and electrode shape and size, because the charge density in the electrode changes first (when the stochastic signal varies) and that change then alters the field in the high field region. Additionally, stochastic heating effects can apply to various locations within the reaction system where there is a sustained electric field, which can include electrode interfaces, as well as liquid-liquid interfaces (such as in vesicles) that are polarized by external fields.

In certain embodiments, the induced field can have a strength of about 10⁷ V/m to about 10⁹ V/m, or about 10⁸ V/m to about 10⁹ V/m at the interface of the target substrate (e.g., the working electrode, a floating electrode, a vesicle membrane, or growing substrate) and reaction system. Additionally, in certain embodiments, the induced field can have a strength of about 10⁷ V/m to about 10⁹ V/m, or about 10⁸ V/m to about 10⁹ V/m in the screening layer adjacent the target substrate. For example, field strengths of about 10⁹ V/m can increase the effective temperature to the aqueous boiling point for 100 amu molecules near flat electrodes. However, smaller field strengths of about 10⁷ V/m (or less) may be sufficient for typical vesicular geometries.

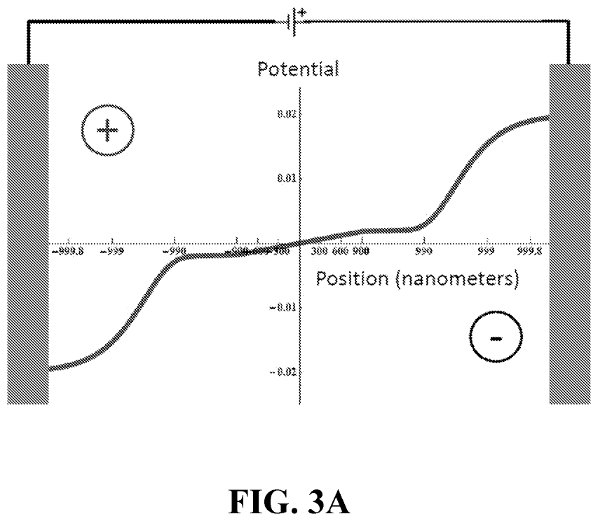

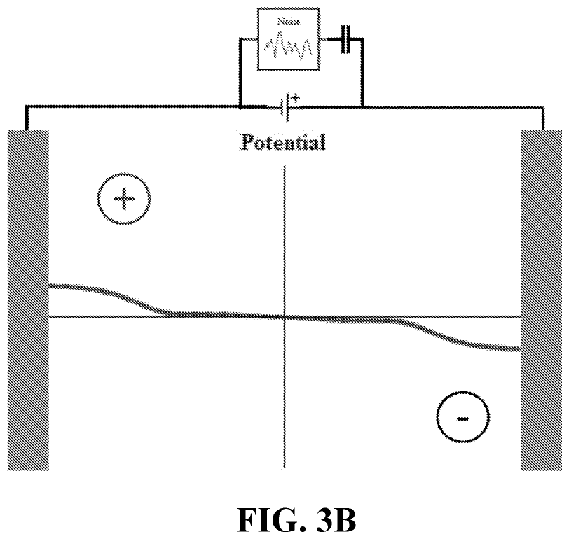

As used herein, the term “screening layer” refers to the layer of reaction system material adjacent the target substrate within which the induced electric field potential is strong enough such that the ionic reactants experience a significant increase in kinetic energy (see FIGS. 3A and 3B). In certain embodiments, the screening layer thickness for a particular field strength may be approximated by the strength of the electric signal and the Debye length of the particular system. For example, when a voltage V₀ is applied across two electrodes immersed in an aqueous, electrolytic solution, the electric field strength E(x) near each electrode may be approximated as

$$E ( x ) ≅ \frac{{V}_{0}}{λ} {e}^{- x / λ} \qquad ( 3 )$$

where λ is the Debye screening length. Thus, for electrolyte concentrations of ˜200 mM (where λ˜1-2 nm), at the electrode-solution interface (where x=0), the field is V₀/λ, which is ˜10⁸-10⁹ V/m for a voltage of ˜1 V. Notably, the electric field strength declines rapidly as the distance increases away from the electrode/substrate surface (e.g., Debye lengths≈1 nm for 0.1 M salt solutions), and thus the stochastic heating effects tend to localize in the screening layers near the electrode or growing substrate interfaces. In certain embodiments, the screening layer has a thickness of about 0.1 nm to about 5 nm, about 0.2 nm to about 3 nm, or about 0.3 nm to about 1 nm.

Without being bound by any theory, it is believed that stochastic heating works because the stochastic field sufficiently invigorates the kinetic motion of molecular charges such that they behave like thermally hot molecules even though the neutral bulk solvent may remain nearly at ambient temperature. Equating the field (E₀)-induced kinetic energy change ΔKE of a charged molecule of mass m to ½ k_{B}ΔT and using the drift velocity ν_{d}=μE₀ to compute ΔKE, it is estimated that the effective temperature elevation ΔT_{eff} of the agitated charge can be

$$Δ {T}_{e f f} = \frac{m {μ}^{2} {E}_{0}^{2}}{{k}_{B}} \qquad ( 4 )$$

where μ is the ion's electrical mobility. Using a typical mobility of μ˜5×10⁻⁸ m²·V⁻¹s⁻¹ for an ion mass of 100 amu, a field of ˜10⁹ V·m⁻¹ can raise the effective temperature by about 80 K (and exceed the aqueous boiling point). As noted above, such fields—1 volt across 1 nm—can be achieved within the Helmholtz screening layers of aqueous electrolytes in which modest ionic strengths (about 0.1 M) lead to screening lengths (screening layer thicknesses) of about 1 nm. Fields of about 10⁹ V·m⁻¹ have been corroborated through Stark effect-based determinations of fields in screening layers. Screening lengths (screening layer thicknesses) in ionic liquids can be even smaller—about 3 Å to about 5 Å.

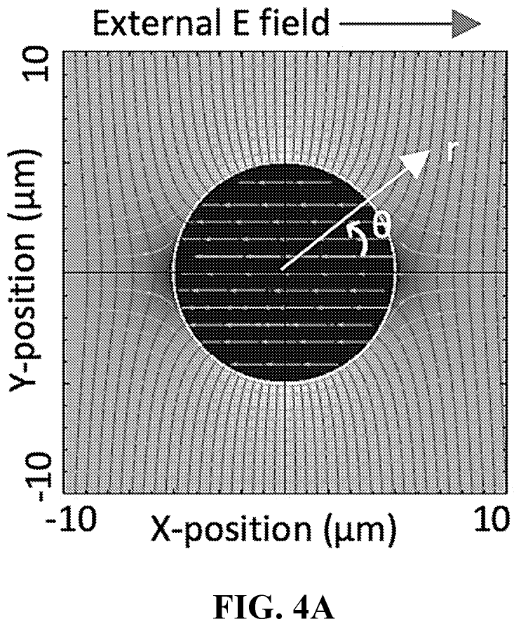

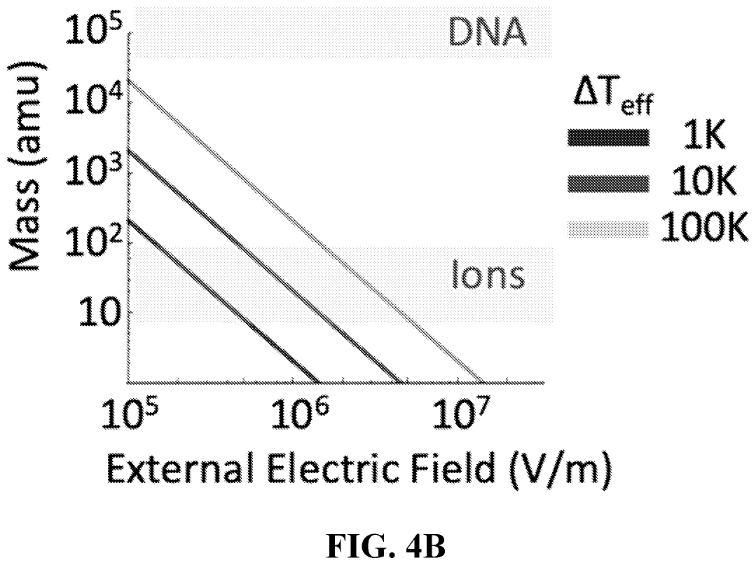

The Arrhenius equation also describes the enzyme kinetics of biological cells. Again, the reaction rate may be amplified by reducing barrier height or intensifying fluctuation strength. Weak electric fields can amplify these kinetics. Transmembrane enzymes, in particular, can be sped up by capturing energy from a weak external field. This process is essentially an electrochemical reaction: the applied field activates a chemical step by reducing the barrier height for that step, not by intensifying the fluctuation strength. Without being bound by any theory, it is believed that a vesicle (or cell) intensifies any externally applied electric field in its membrane and interfacial regions due to its geometry. FIG. 4A presents a theoretical picture of this effect for a 10 μm vesicle with a d≈5 nm-thick membrane obtained by solving Poisson's equation. The potential drops sharply across the membrane region so the radial field is very large:

$${\vec{E}}_{r} ≅ \frac{1}{2} {E}_{0} ( \frac{{r}_{V}}{d} ) cos θ \hat{r} \qquad ( 5 )$$

where the vesicle radius r_{V}=5 μm. Because

$$\frac{{γ}_{V}}{d} ≅ 1 {0}^{3} ,$$

the radial field at the poles of the vesicle is very sensitive to the applied field E₀. The three profiles in FIG. 4B demonstrate the molecular masses and field strengths necessary to realize effective temperature amplifications of 1 K, 10 K, and 100 K in the field-enhanced region of a vesicle (i.e., Egn. 4, re-written as

$$m = \frac{4 {k}_{B}}{{{μ}^{2} ( \frac{{r}_{V}}{d} {E}_{0} )}^{2}} Δ {T}_{e f f} ) .$$

Vesicular geometry requires considerably smaller applied fields than flat electrodes (e.g., about 10⁷ V·m⁻¹, as compared to versus about 10⁹ V·m⁻¹ for a 100 amu molecule to have a 100 K increase in temperature).

Stochastic heating methods in accordance with embodiments of the present invention may be used in a variety of applications and industries. Such applications include, but are not limited to, light weight coatings for AM parts, electro-catalysis, coating capsule interiors, corrosion coatings, catalysis synthesis and processing science, and applications in biosciences.

Embodiments of particular applications are described in greater detail below. However, it should be understood that these applications do not necessarily limit the overall scope of the invention. Additionally, certain features described with respect to one embodiment are not necessarily mutually exclusive to other embodiments, and thus such features described with respect to one embodiment may also be used in conjunction with other embodiments.

### Electrodeposition

Many industrially significant electrochemical processes—including the electro-plating of heavy metals; electro-deposition in non-aqueous solvents; and electrochemical recovery methods—suffer from kinetically limited reaction rates. Unfortunately, the efficacy of raising the temperature to address this problem is fundamentally limited by the boiling point of the solution. Therefore, speeding up electrochemical reactions while maintaining near ambient bath temperatures is an important advancement for electrochemical processes. Accordingly, methods in accordance with embodiments of the present invention may be useful for thin film deposition, such as metal deposition (e.g., gold, silver, etc.), including magnetic metal materials, and conducting polymer deposition (e.g., poly[ethylenedioxythiophene](PEDOT) polymer films).

In certain embodiments, stochastic heating methods described herein may be used in an electrodeposition process. In particular embodiments, the electrodeposition process may comprise growing films of metals, such as electroplating or the formation of anti-corrosion coatings. In certain such embodiments, the one or more ionic reactants can comprise metal cations of gold (Au), silver (Ag), Copper (Cu), and/or chromium (Cr), which may be derived from metal-containing salts and compounds (e.g., AuCl, AgNO₃, etc.).

In certain embodiments, the methods described herein may be used in chromium (III) (Cr(III)) deposition, which is much safer than the carcinogenic Cr(VI) processes. Thus, stochastic heating methods may be used in Cr³⁺ electroplating, which is conventionally limited by the slow kinetics of the [Cr(H₂O)₆]³⁺ complex.

Other applications may include rare earth metal deposition (e.g., for ultra-strong magnets on circuit boards) and fast deposition from non-aqueous solvents.

### Chemical Reactions

Purely chemical reactions may also be enhanced by stochastic heating methods according to embodiments of the present invention. In particular, reaction rates and growth-rates can be amplified (i.e., increased) in chemical reactions without heating the overall reaction systems. In precipitation reactions, the stochastic heating methods can be used to increase the nucleation rate, the growth rate, or both. The stochastic signal applied, and thus the generated field, can be varied depending on whether nucleation or growth is preferred. Exemplary precipitation reactions can include the production of calcium carbonate (CaCO₃), magnesium carbonate (MgCO₃), calcium phosphate (CaHPO₄), zinc oxide (ZnO), and copper sulfide (CuS).

### Biological Processes

Embodiments of the present inventions extend beyond classical electrochemical or chemical applications. A basic element of the present invention is the use of high field regions where charged molecules will swiftly and strongly respond to the external stochastic field. This situation also arises in biological cells and vesicles. Therefore, when exposed to a stochastic field, the thin mantle of solution inside a cell or vesicle that borders the membrane on the up-field and down field poles of the cell similarly experiences a strong stochastic field. As a consequence, the charged species in those regions can experience enhanced kinetic activity and an effectively elevated temperature. Thus, the chemical reactivity can be enhanced. As many cellular species are charged, including a significant fraction of the membrane bound enzymes that are permanently located in high field regions, stochastic heating may provide a unique mechanism to control important cellular processes (e.g., ATP generation, pH level (H⁺ pumping), DNA replication). In particular, by populating the vesicular membrane or cytoplasm with reactive molecules, such as enzymes or DNA, the rates of enzymatic turnover rates may be controlled by controlling the exogenous stochastic field. Thus, in certain embodiments, applying a stochastic signal to reaction systems, as described herein, may amplify the transport rate of enzymes or other charged atoms and molecules across a vesicular membrane and/or amplify the rate of biological processes occurring within the vesicle or biological system.

In particular embodiments, a stochastic heating method may be used to activate the Polymerase Chain Reaction (PCR) in lipid vesicles in order to amplify their internal DNA copy numbers in a quasi-athermal manner. The method generally comprises that following steps: 1) DNA trapping; 2) stochastic denaturation; 3) annealing; and 4) stochastic elongation.

The reaction system generally comprises lipid vesicles dispersed in a buffered PCR solution. Exemplary lipid vesicles may comprise artificially phospholipid vesicles, such as those comprising 1-palmitoyl-2-oleoyl-sn-glycero-3-phosphocholine (POPC) and may comprise DNA molecules contained within the cytoplasm fluid of the vesicles. The PCR solution may be an aqueous solution, such as a commercially-provided solution for PCR, and may further comprise phospholipids (e.g., 1,2-Distearoyl-sn-Glycero-3-Phosphoethanolamine (DSPE) conjugated Polyethylene Glycol (PEG)), cholesterol, trisaminomethane buffer, primer(s), DNA template(s), and DNA dye(s). Various enzymes may also be included in the reaction system or added later in the process. In certain embodiments, the method may utilize an initial reaction system temperature of about room temperature (about 15° C. to about 25° C., or about 20° C.), which can serve as the base temperature for the system throughout the stochastic PCR process. However, in certain other embodiments, the initial reaction system temperature may be about 50° C. to about 60° C., or about 55° C., such that no stochastic signal is required for the annealing step.

The DNA trapping step comprises generating a signal with electronic signal generation equipment and transmitting the signal to a working electrode disposed within the reaction system, thereby generating an electric field within the reaction system. The field is particularly strong at the vesicle membranes, and thus the negatively charged DNA molecules become interfacially trapped at the screening layer near the membrane (e.g., within about 5 nm, or less) within the vesicles.

The stochastic denaturation step comprises applying noise (i.e., a stochastic signal) to effectively “heat” the interfacially trapped ds-DNA molecules at the membrane. The field strength and noise can be adjusted so as to provide an “effective temperature” in the screening layer of about 90° C. to about 100° C., or about 95° C., which is the conventional system temperature required to denature the DNA.

After denaturation, DNA annealing may be induced by setting the stochastic signal to a level so as to provide an “effective temperature” in the screening layer of about 50° C. to about 60° C., or about 55° C., which is the conventional system temperature required for annealing. However, when the reaction system initial temperature is about 50° C. to about 60° C., or about 55° C., annealing may be induced by simply by turning off the stochastic signal, thereby returning the “effective temperature” in the screening layer to the initial/base temperature of the reaction system, since annealing occurs spontaneously at about 55° C.

After annealing, elongation may be induced by setting the stochastic signal to a level so as to provide an “effective temperature” in the screening layer of about 70° C. to about 75° C., or about 72° C., which is the conventional system temperature required for elongation. Additionally, or alternatively, the entire reaction system can be heated to the above-noted temperatures to complete the elongation step.

The stochastically-assisted PCR method described herein can advantageously occurs with little or no thermal cycling (i.e., heating/cooling the reaction system) required in traditional PCR processes. Thus, in certain embodiments, the method does not comprise heating the reaction system above 80° C., or above 90° C. In certain embodiments, the method comprises an isothermal PCR method. In certain embodiments, the isothermal PCR method occurs at (i.e., the reaction system temperature is maintained throughout the process at) about 50° C. to about 60° C., or about 55° C. In certain embodiments, the isothermal PCR method occurs at (i.e., the reaction system temperature is maintained throughout the process at) about 15° C. to about 25° C., or about 20° C., or about 25° C.

### Other Considerations

Additional advantages of the various embodiments of the invention will be apparent to those skilled in the art upon review of the disclosure herein and the working examples below. It will be appreciated that the various embodiments described herein are not necessarily mutually exclusive unless otherwise indicated herein. For example, a feature described or depicted in one embodiment may also be included in other embodiments, but is not necessarily included. Thus, the present invention encompasses a variety of combinations and/or integrations of the specific embodiments described herein.

As used herein, the phrase “and/or,” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

The present description also uses numerical ranges to quantify certain parameters relating to various embodiments of the invention. It should be understood that when numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

### EXAMPLES

The following examples set forth methods in accordance with the invention. It is to be understood, however, that these examples are provided by way of illustration and nothing therein should be taken as a limitation upon the overall scope of the invention.

### Example I

### Gold Electrodeposition

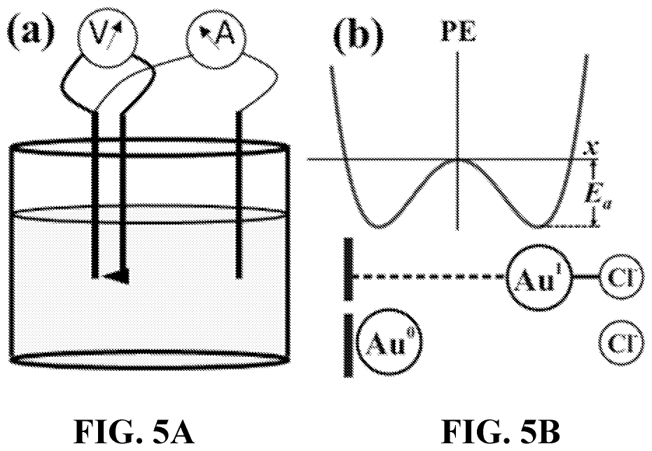

Given the importance of Arrhenius behavior in chemistry, this example is a study that extended Martinez's macroscopic simulant of a chemical reaction to a molecular system. The study focuses on the electrochemical reaction for gold deposition:

$${Au}^{I} {Cl}_{Ad} + {e}^{-} {↔}^{k , {k}_{-}} {Au}^{0} + {Cl}^{-} ,$$

where Ad denotes an electrode-adsorbed species. This reaction occurs in aqueous K⁺(Au^{III}Cl₄)⁻ solution when a voltage is applied between electrodes in the solution (FIG. 5A).

The model shown in FIG. 5B adapts the theory for stochastic heating to elucidate how external noise affects the Au^{I}→Au⁰ reaction. The upper panel illustrates this reaction, showing an Au^{I} ion of charge e adsorbed to the electrode surface. The Au^{I} bond to the surface has force constant κ. x(t) is the reaction coordinate, representing the ion's distance from the electrode. The surrounding solution has a viscous friction coefficient of α. The curve denotes the potential energy (PE) of the gold atom as a function of x, the right (left) well of which corresponds to Au^{I} (Au⁰). The inter-well barrier height, which represents the activation energy Eₐ, reflects the energy required for Au^{I} to transition to Au⁰ by breaking its bond with Cl⁻, migrating to the surface, and accepting a charge e from the electrode (lower panel). Stochastic heating is the enhancement of the temperature T_{eff} by applying an external random force f_{N} that acts selectively on the reactants in the system. f_{N}, which is characterized by a both a strength Γ and a bandwidth Δf, adds to the ubiquitous Brownian force of the bath f_{B}. In the case where f_{N} has zero-mean and is perfectly white (i.e., its bandwidth Δf→∞,) the total random force on a charged molecule is simply f_{B}+f_{E}. The fluctuation-dissipation theorem, which relates the random force on a particle to its temperature, then predicts that the agitated reactant molecules will have an effective (kinetic) temperature T_{Eff} greater than that of the surrounding bath T. In the case where f_{N} is not perfectly white,

$${T}_{E f f} = T + \frac{{Γ}^{2}}{( κ + α γ ) {k}_{B}} [ 1 - {e}^{- ( γ + κ / γ ) t} ] \qquad ( 6 )$$

where γ∝Δf. Eqn. (6) shows that the noise signal is most effective when the noise strength Γ is large and the bandwidth Δf is broad. When Δf→∞, Eqn. (6) reduces to Eqn. (2).

The Butler-Volmer (BV) equation is an Arrhenius equation that describes electrochemical reactions:

$$j ∝ exp [ - \frac{( {E}_{a} + e η )}{2 {k}_{B} T} ] \qquad ( 7 )$$

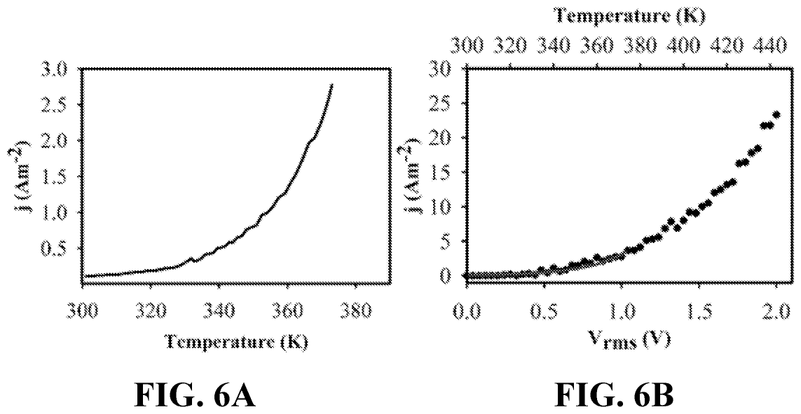

FIG. 6A shows the Au^{I}→Au⁰ reaction rate (expressed as current density j) observed when the temperature is increased from 300 K to 376 K while applying a small overpotential (η=−20 mV). As the BV equation predicts, j rises as predicted by Eqn. (7) but cannot exceed ˜2.5 A m⁻² because the maximum temperature that can be attained is limited by the aqueous boiling point. In contrast, FIG. 6B shows the reaction rate when a stochastic voltage-signal is added to the overpotential. The rate (filled circles) rises exponentially as the noise-amplitude is raised, but T stays below ˜303 K (as determined by an in situ temperature probe). Much larger current densities are attainable: ˜25 A m⁻². Thus, noise can drive j an order of magnitude higher than can T. Fitting this curve to the thermal data of FIG. 6A (line overlaying circles in FIG. 6B) shows that current densities can be induced commensurate with T˜440 K. Other factors being equal, such large current densities would be impossible to achieve through conventional heating due to the boiling point of the solution. It is believed these data are the first demonstration of electrochemical stochastic heating.

### Example II

### Silver Electrodeposition

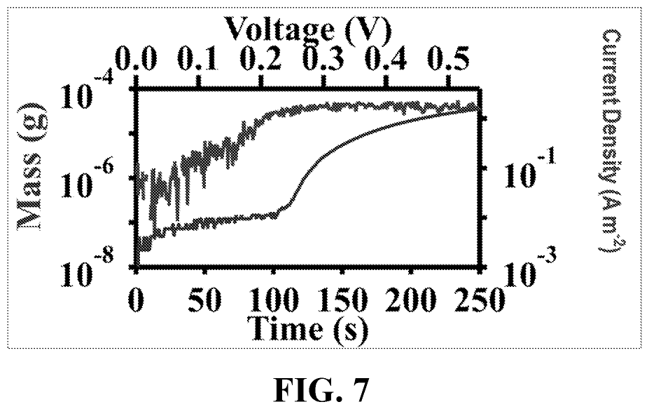

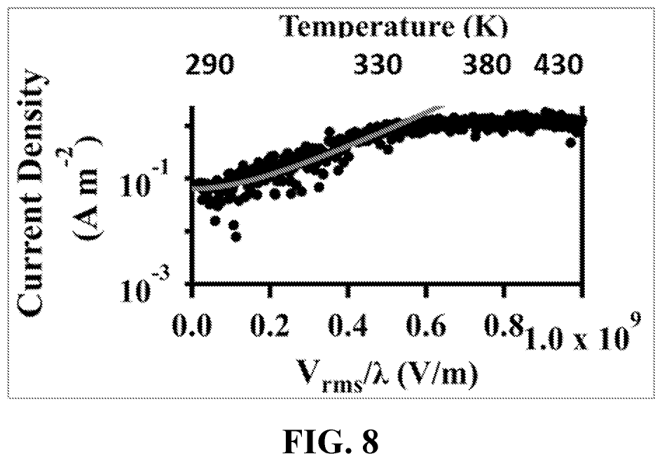

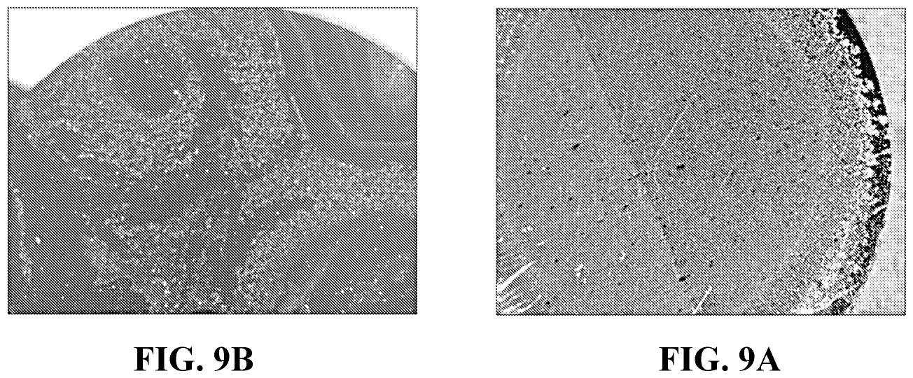

This example applied the gold electrodeposition principles set forth in EXAMPLE I to a silver electrodeposition experiment. The reaction solution contained 5 mM AgNO₃ and 200 mM KNO₃. The working electrode and the counter electrode were both gold. The reference electrode was a silver wire. Using a quartz crystal microbalance, the deposition rate with and without stochastic noise was repeatedly measured over a period of 250 seconds (see FIG. 7). The use of stochastic noise was shown to increase the deposition rate by ˜21× amplification as compared to the no noise sample. The effective temperature at different field strengths for this system is shown in FIG. 8. Over the measured time period, the noise sample showed evidence of silver deposition (FIG. 9A), while the no noise sample showed no deposition (FIG. 9B).

### Example III

### Calcium Carbonate Precipitation

The demonstration of stochastically driven enhancement of gold and silver deposition rates is very promising but is complicated by overlaying two electric fields: a constant field that drives the reaction forward and a stochastic field that adds kinetic energy to the ions. As a simpler system, the growth-rate enhancement of a purely chemical reaction is demonstrated: calcium carbonate precipitation (Ca²⁺CO₃²⁻→CaCO₃).

The reaction solution contained 3 mM of CaCl₂ and NaHCO₃. The pH was 8.12, and the temperature was 21.0° C. Q/K=12. Noise strength=700 mV (rms).

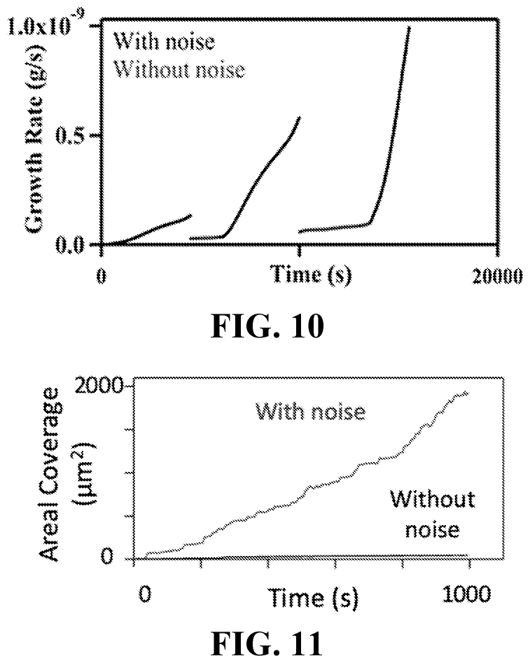

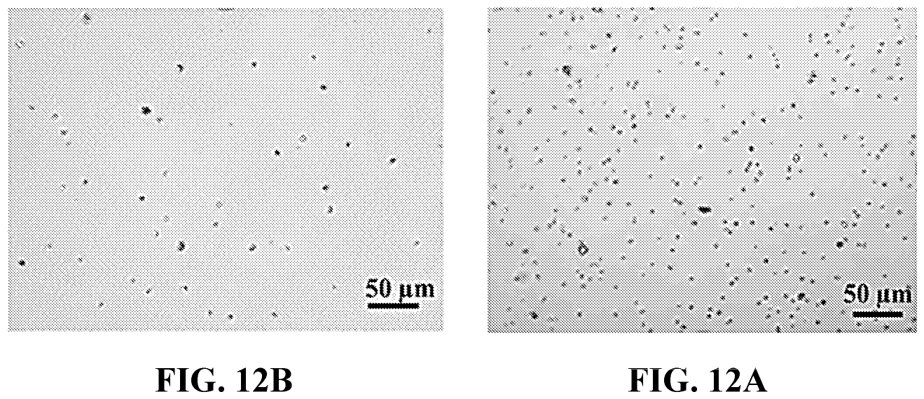

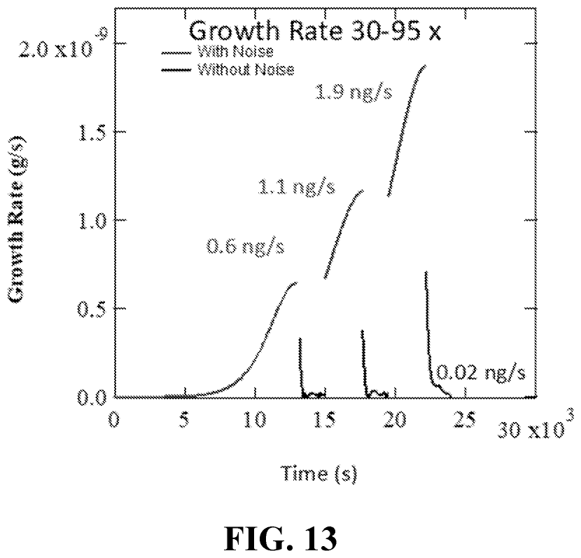

Using a quartz crystal microbalance, the growth rate (FIG. 10) and areal coverage (FIG. 11) with and without stochastic noise was repeatedly measured. Nucleation differences are shown in FIG. 12A (with stochastic noise) and FIG. 12B (without stochastic noise). Additionally, the use of stochastic noise was shown to increase the growth rate by 30-95× compared to the use of no noise (see FIG. 13).

### Example IV

### Nucleation and Growth

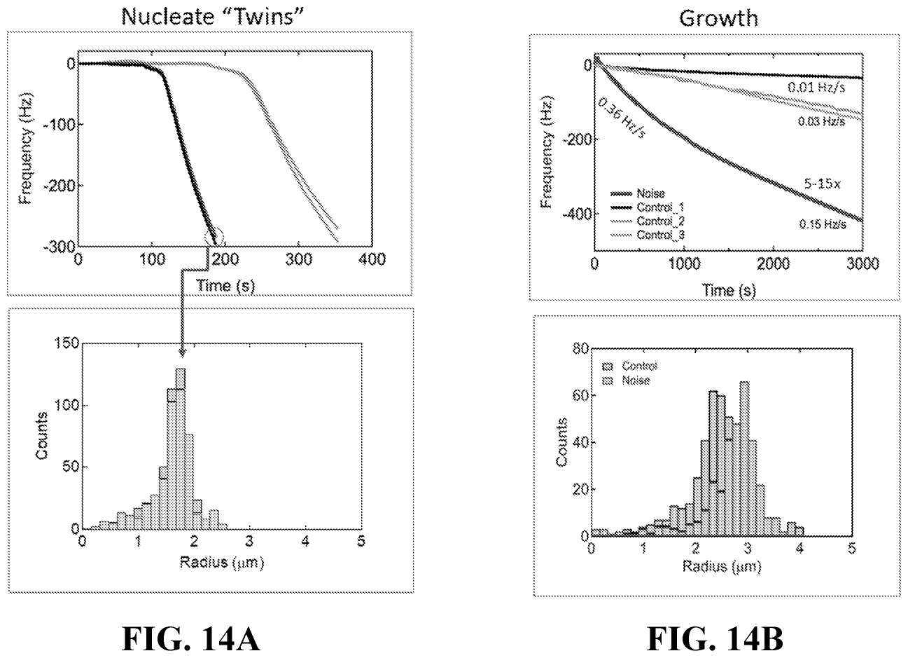

A variation of EXAMPLE III was performed to exploit pH-sensitivity and examine differences in nucleation and growth rate amplification compared to direct electro-precipitation. The reaction solution contained 4 mM CaCl₂, 4 mM NaHCO₃, and 200 mM NaCl. The pH was 8.1. Q/K=6.2. The nucleation rates are shown in FIG. 14A. The growth rates are shown in FIG. 14B. ˜10× amplification was shown with the noise applied.

Notably, stochastic heating can lead to calcite-growth without acidification of the solution (unlike direct electro precipitation methods), as this route does not rely on oxygen-reduction.

### Example V

### Magnesium Carbonate Precipitation

A variation of EXAMPLE III was performed to grow magnesite. Magnesite is a buffered system where pH is less of a concern. Thus, stochastic heating for magnesite growth has promising chemistry for ex situ CO₂ fixation. However, magnesite precipitation traditionally as notoriously slow kinetics. The active area of the electrode was coated with a hydrophobic polymer to promote deposition. The reaction system contained 0.25 M of MgCl₂ and 0.3 M of (NH₄)₂CO₃. The pH was 7.0. Q/K>>10.

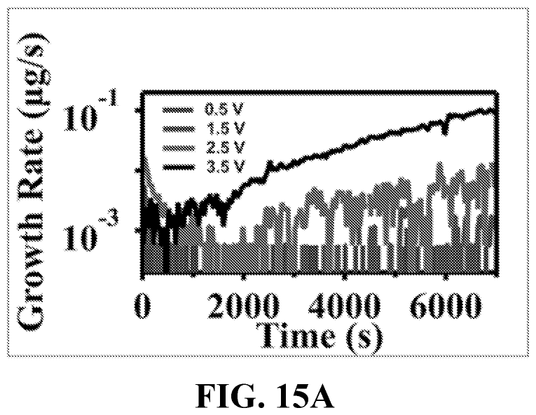

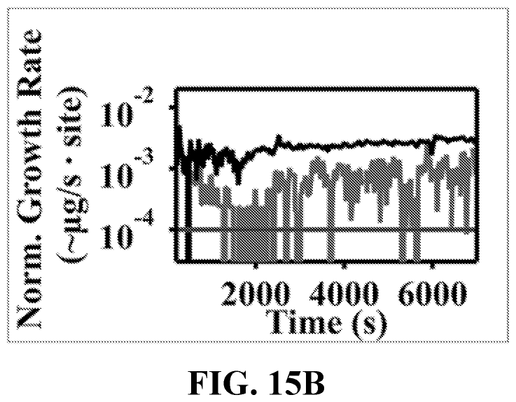

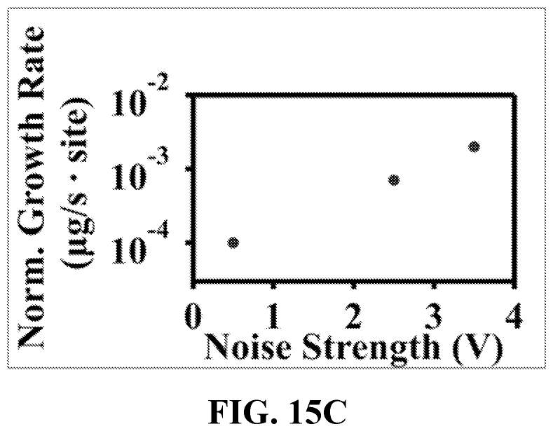

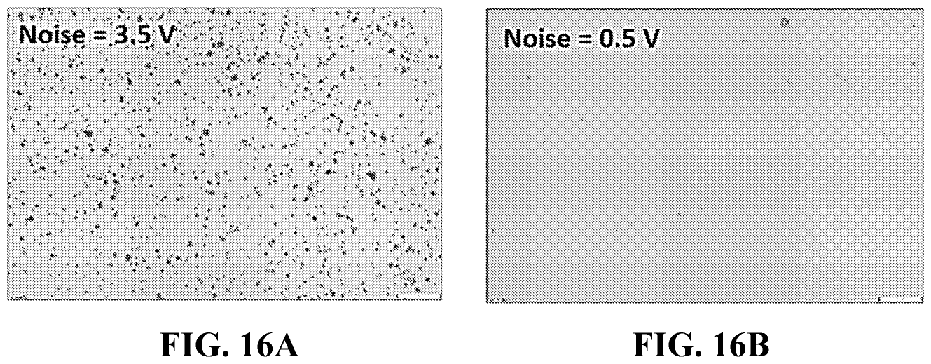

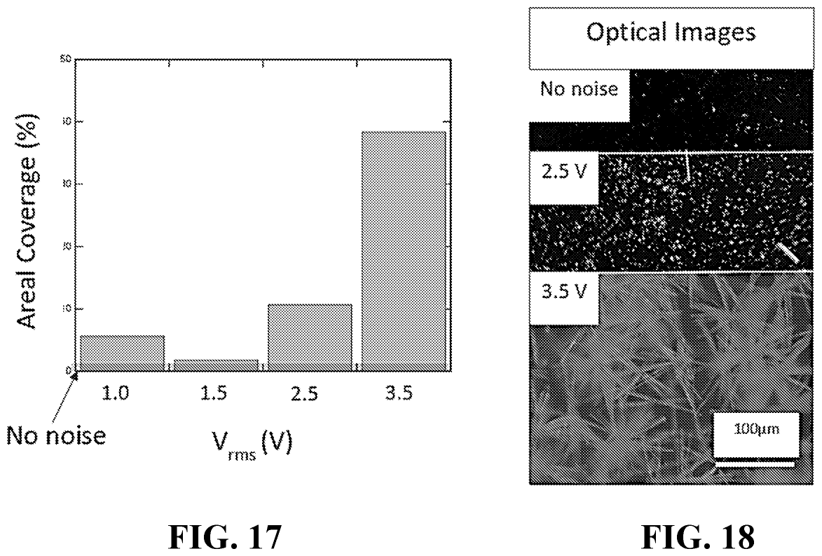

Voltages of 0.5 V, 1.0 V, 1.5 V, 2.5 V, and 3.5 V were tested, where noise switched on at time-zero. As shown in FIGS. 15A-15C, significant amplification of the growth rate was shown with noise above ˜1.5 V. FIG. 16A and FIG. 16B show the difference in magnesite deposition after 7000 seconds with applying 3.5 V (FIG. 16A) and with applying 0.5 V (FIG. 16B). FIG. 17 shows areal coverage and FIG. 18 shows exemplary optical images of the magnesite after no noise and applying noise for 2 hours. Although higher voltages showed large rate increases, even at low voltages MgCO₃ showed higher growth rates when noise was applied.

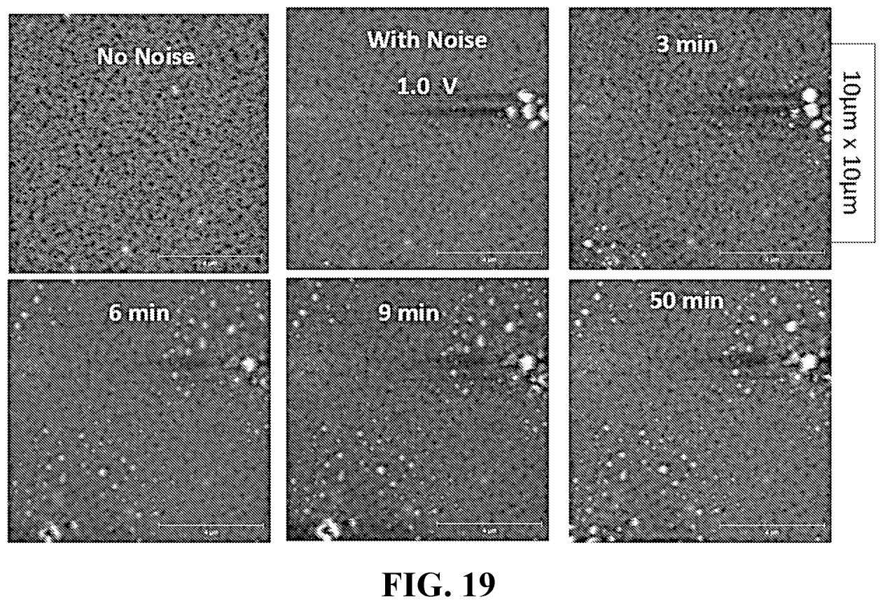

Additionally, there was no evidence of nucleation after 15 minutes at V_{RMS}<0.5 V. Nucleation was evident within 30 seconds at V_{RMS}=1.0 V. FIG. 19 shows the progression of nucleation at V_{RMS}=1.0 V over time. The clustering suggests heterogeneous nucleation.

### Example VI

### Copper Sulfide—Hydrothermal Growth

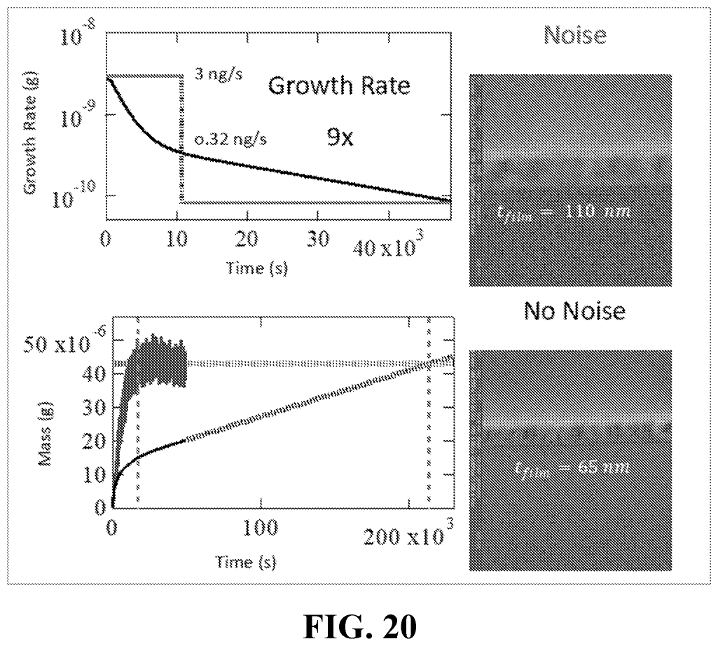

A variation of EXAMPLE III was performed to grow copper sulfide. Copper sulfide is self-limiting, requiring different metrics. The reaction solution contained 3 mM CuSO₄, 3 mM thioacetamide C₂H₅NS, and 1.5 mM EDTA. A noisy stochastic signal was applied at 600 mV (rms). FIG. 20 shows the growth rate and total mass deposited over time. When noise was applied, a growth rate amplification of ˜9× was seen compared to no noise applied.

### Example VII

### Zinc Oxide Nanowires

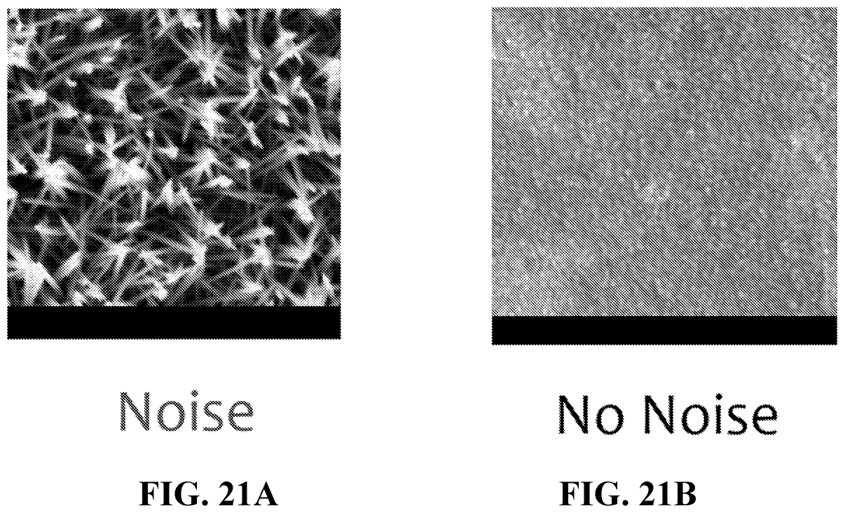

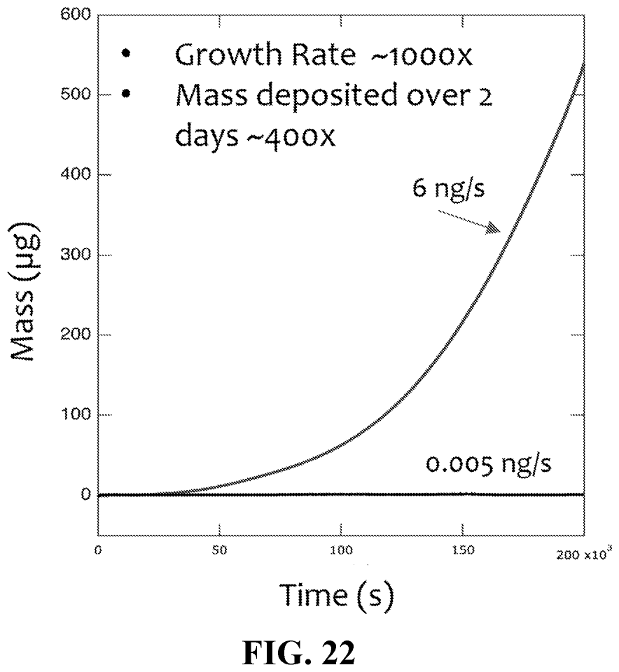

Building off of EXAMPLE VI, zinc oxide nanowires were grown using a similar stochastic heating method. A noticeable increase in growth was seen after 2 days when noise was applied (FIG. 21A) compared to no noise applied (FIG. 21B). As shown in FIG. 22, the growth rate was amplified by ˜1000× and total mass deposited over 2 days was increased by ˜400×.

### Example VIII

### Brushite Crystallization (Prophetic)

Reactions that result in crystal formation permit rate-quantification. Brushite crystallization (Ca²⁺+HPO₄²⁻+2H₂P→CaHPO₄·2H₂O) is a model system associated with bone mineralization. Brushite crystallization depends on supersaturation, calcium-to-phosphate ratio, and ionic strength (which can be calculated from geochemical models). Solution and noise conditions will be monitored using an in situ quartz crystal microbalance (QCM), which measures the total deposition rate (i.e. both nucleation and growth), and in situ AFM to directly measure atomic step kinetics. Simulations will be used investigate attachment and detachment rates of (solvated) Ca⁺⁺ and HPO₄⁻ ions onto single crystallographic steps. These rates are part of the kinetic coefficient for the growth process. Such simulations have been shown to be facile enough to capture these dynamics, permitting meaningful comparison to the AFM step kinetics-measurements. The deposition rate in the presence and absence of stochastic (and other) fields will be monitored using computational and experimental methods. Both QCM and AFM have been modified to introduce electric noise fields at the electrode surface. Similar noise fields will be introduced into the MD simulation. Noise fields may increase growth rates and step kinetics by energizing the ions that participate in nucleation and growth. Interestingly, the stochastic approach differs from simply increasing the temperature because the neutral mineral (CaHPO₄·2H₂O) is not affected by the electric field and thus its solubility product does not change. Hence, higher effective temperatures and faster growth rates may occur upon application of noise.

### Example IX

### Bismuth Vanadate Growth (Prophetic)

The primary goal of this example is to further demonstrate using stochastic heating methods for the growth of materials at ambient conditions that could normally only be grown above the boiling point of water. The central physics question that this example may answer is whether adding kinetic energy to the system (which increases momentum transfer) induces the same chemistry as increasing the temperature (which increases both momentum transfer and the vibrational energy distribution of the molecules). Growing materials at room temperature may demonstrate that stochastic heating enables in situ investigations that would previously have required specialized hydrothermal cells. Rapid temperature profiles will be demonstrated, which cannot be achieved in conventional systems due to their large thermal mass. An advantage of this last process will be to enable direct control over the local supersaturation by rapidly turning on and off the release of ions from chelating molecules. This would confer a level of control that is currently unavailable for solution crystal growth.

The testbed will be the growth of BiVO₄ due to its relative simplicity and current technological interest, but most importantly because it exemplifies a large class of materials that are grown by decomposition of organic-metal complexes. The chelating agent ethyl enediaminetetraacetic acid (EDTA) complexes with many 2+ and 3+ charged ions and then releases them as a function of temperature (typically above 100° C.). For this reason, it is a common component of many hydrothermally processed materials such as perovskites, biominerals and oxides that have broad applications including photovoltaics, ionic conduction, ferroelectric materials, electrocatalysts and photocatalysts.

### Example X

### Action-at-a-Distance Stochastic Heating: Proof-of-Principle

This example demonstrates that chemical reaction rates can be enhanced at unwired, electrically floating electrodes. Without being bound by any theory, it is believed that fluctuating electric fields form at the floating interfaces, stochastically heating the ions in these screening layers.

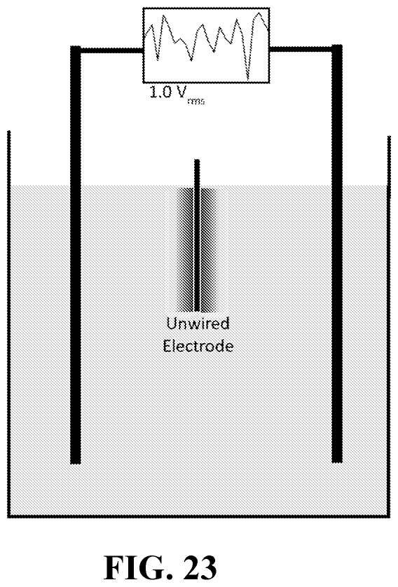

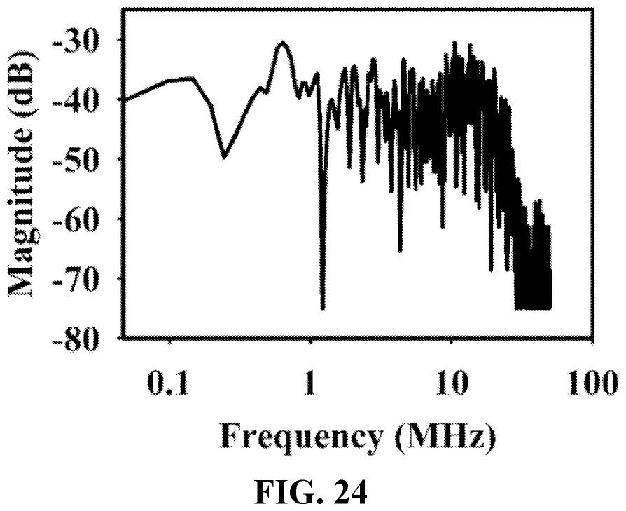

To demonstrate the feasibility of this process, a weakly saturated aqueous solution of CaCl₂) and NaHCO₃ was prepared. In the absence of stochastic excitation, the Ca²⁺+CO²⁻→CaCO₃ reaction will produce calcite crystals on a slow (days-to-weeks) time-scale. However, in the presence of a stochastic field, the field accelerates calcite-crystallization on the wired electrodes. Here, this process is investigated on unwired electrodes. A small unwired (gold) electrode was mounted between two large, wired electrodes (See FIG. 23). In operation, it is believed that the stochastic field (bandwidth shown in FIG. 24) polarizes the small, unwired electrode and established screening layers at its interfaces much faster than at the large, wired electrodes. Hence, the fluctuating field was expected to stochastically heat the ions in these screening layers. The reaction parameters included: 4.0 mM CaCl₂) and 4.0 mM NaHCO₃ in an aqueous (water) solution, with a pH of 8.07 and a temperature of 293 K. 1.0 V_{RMS} signal was initially applied.

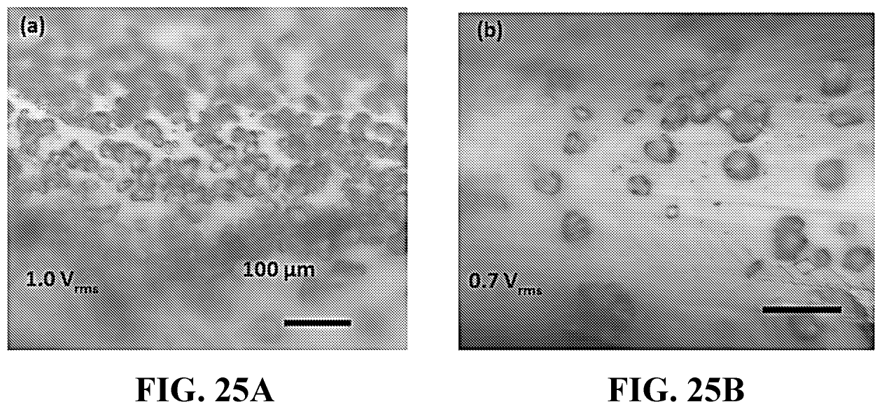

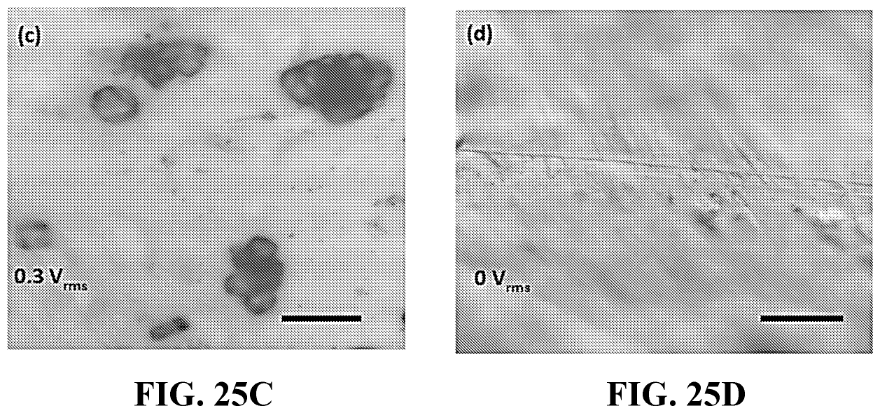

After a 12 hr period of 1.0 V_{RMS} excitation, the unwired electrode was densely covered with crystallites, as the optical micrograph in FIG. 25A shows. The solution-temperature was still 293 K. Reduced excitation (0.7 V_{RMS} and 0.3 V_{RMS}) induced less crystallization (see FIG. 25B and FIG. 25C, respectively). In the control experiment with no stochastic excitation (FIG. 25D), the unwired electrode showed no crystallization. These observations indicate that the stochastic field induces significant crystallization on this time-scale.

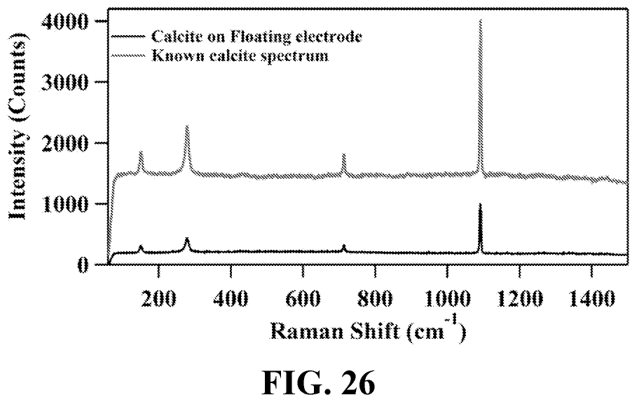

The Raman analysis (see FIG. 26) confirms that the deposit on the unwired electrode is calcite. The Raman spectrum from the control electrode (not shown) was featureless. Thus, stochastic fields are effective at amplifying chemical rates at electrically floating electrodes.

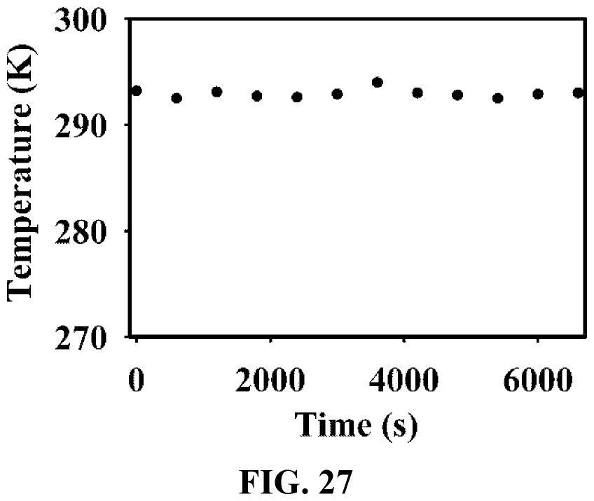

One concern is that the noise-field may elevate the temperature of the unwired electrode to a significant degree. To investigate this possibility, the floating, gold electrode was replaced with a Type K (chromel/alumel) bead-wire thermocouple. Due to its metallic nature and small size, the bead should respond similarly to the stochastic field as the floating electrode. FIG. 27 plots the temperature versus time over a ˜2 hour period of 1.0 V_{RMS} stochastic excitation. The temperature is essentially steady (to within ±0.4 K) over the observation-period. This finding suggests that the stochastic field does not significantly heat up the gold electrode in the unwired geometry.

### Example XI

### DNA Melting (Prophetic)

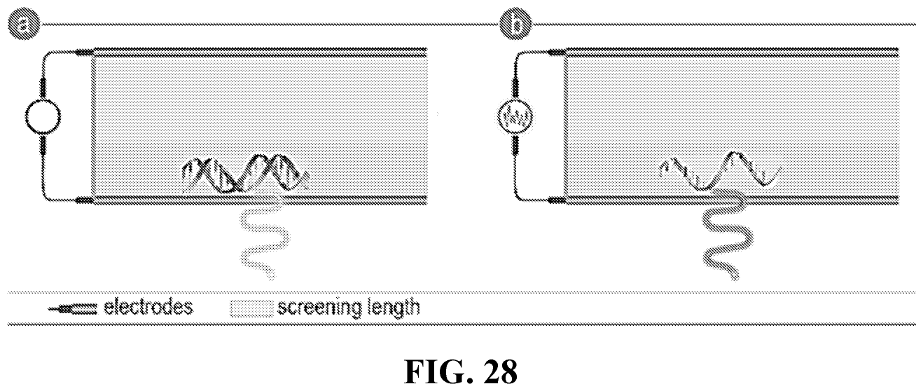

DNA melting is an example of a thermally driven reaction. The large mass (˜660 amu/bp), high charge, and well-developed set of fluorescence markers for DNA make it a good stochastic heating candidate. The approach will be to use a constant field to trap double-stranded DNA (ds-DNA) near a transparent ITO electrode surface (FIG. 28) and then use stochastic fields to raise the effective temperature to ˜95° C., where DNA melts. To maximize the ability to detect interfacial fluorescence and discriminate against far field background, an inverted total internal reflection fluorescence (TIRF) microscope for fluorescence detection will be used. A DC voltage will be applied across the electrodes to attract and trap DNA at the interface of the lower electrode and detect trapping by observing an increase in fluorescence intensity. SYBR Green I is a dye with a high specificity for double-stranded DNA (dsDNA). Next, the stochastic field will be applied to induce melting. The QuantiFluor ss-DNA Dye System is a dye for single-stranded DNA (ssDNA). ssDNA fluorescence along the interface of the trapping electrode will be expected. From the time-dependent fluorescence, the melting kinetics can be characterized, as well as calibration the noise-strength to the melting temperature.

The effect of stochastic heating on DNA melting near an interface will be studied using MD simulations employing the AMBER force field. Simulations of a 25 bp sequence (two full turns) of DNA will be performed in explicit solvent with counter ions and bulk salt. The effect of stochastic fields on the melting of DNA will be monitored by measuring the root mean square deviation from a canonical DNA geometry during the simulations. While full denaturation is unlikely to occur due to the long times involved, it may be possible to follow signs of the initiation of melting with reasonably long (10 μs) simulation times.

### Example XII

### Stochastically Amplify Vesicular Reaction Rates (Prophetic)

There is substantial interest in new methods for amplifying reaction rates in confined spaces such as vesicles, cells, droplets, and emulsions. This example aims to establish such a method, using stochastic heating. This process will be demonstrated using two vesicular systems. The potential near a vesicular membrane containing free ions is similar to that near the detached third substrate. Geometric arguments indicate an electric field enhancement of

$$\frac{radius}{thickness} ≅ {10}^{3}$$

in the screening layer of vesicular membranes. Thus, it is believed that external stochastic fields will kinetically excite charged ions in these screening layers and thereby amplify their reaction rates. This concept will be tested using brushite nucleation/growth and DNA melting. MD simulations will be used to estimate spatially dependent field enhancements and ion kinetic energies near the membrane interface. The significance of this example is that it further demonstrates the ability to control reactions within floating micro-reactors such as vesicles, cells, droplets, capsules, or emulsions, which are of widespread fundamental and industrial interest.

To test whether mineralization can be induced within vesicles using smaller fields than are needed for flat electrodes, nucleation rates of supersaturated brushite solution using will be measured. Solution filled microcapsules suspended in oil (octane) will be arranged in an approximately 2D array between ITO electrodes. Large numbers of homogeneously-sized vesicles are needed for a statistical analysis of nucleation. For this reason, silicone microcapsules that were developed for CO₂ sequestration will be used. Capsules with 600 μm diameter and 30 μm thickness may increase the field strength by an order of magnitude. Imaging confocal Raman microscopy will be used to monitor the phosphate vi peak that shifts from −950 to 985 cm¹ depending on the calcium phosphate phase.

Applying a stochastic field to DNA-containing vesicles may melt the double-stranded DNA, producing single-stranded DNA at the vesicular interfaces. To test this, the vesicular interfaces for ssDNA number-growth during stochastic excitation will be examined. This experiment will be performed on the TIRF microscope. As it is estimated ˜3-30 dsDNA molecules in the illuminated interfacial volume at a given time, the high fluorescence sensitivity and background rejection of this instrument are ideal. Single molecule fluorescence detection of ssDNA and dsDNA has been demonstrated. After attracting DNA to the lower vesicular interfaces, the stochastic field will be applied to heat the molecules and induce melting. Single stranded DNA will be detected fluorescently, as above. The TIRF optics will be tuned so that the illumination depth contains the inner vesicular interfaces. The significance of this example is that observation of single-stranded DNA will indicate stochastically induced, intra-vesicular DNA melting.

Simulations of the effects of stochastic fields on the heating of ions inside vesicles will be performed. The presence of a lipid membrane will modulate the electric field within the vesicle when placed between two electrodes. Differences in heating behavior due to the membrane will be investigated. This represents a rather large system (roughly 10×10×25 nm³). Contributions from the ions and the ionic atmosphere will be isolated by determining the local effective temperature.

### Example XIII

### PCR in Lipid Vesicles (Prophetic)

The field of electrochemistry is based on using voltages to vary the activation energy of electrode-enhanced reactions, thereby controlling their rates. A similar feat will be done with biological cells. For example, it is an experimental fact that external electric fields affect the transport rates of membrane bound-enzymes in biological cells. Previous studies have investigated the effect of weak electric fields on biological cells. Because external electric fields are intensified in and near the membranes of biological cells, weak fields can amplify the activity of membrane-bound enzymes that have charged moieties. A previous study demonstrated this effect by showing that enzymes responsible for ferrying substrates into a cell speed up by capturing energy from an external electric field. Hence, this process is essentially an electrochemical reaction: the applied field activates a chemical step by reducing the barrier height for that step.

The de novo design of artificial cells faces the challenge of producing vesicular compartments that contain functional cell components—ribosomes, mitochondria, molecular machines, etc.—and that are also capable of dividing into viable daughter cells that closely resemble their progenitor. All of these functions rely on chemical activity. Biologically, cells employ enzymes to activate their chemical machinery. Physically, however, we understand that temperature also dictates biochemical reaction rates (Arrhenius behavior). Having temperature as a working parameter in synthetic cellular design would be useful because much biochemical skill relies on thermal control. However, the temperature range in which most living systems remain viable is not very large. The fundamental limitations are that water itself is liquid only between 0-100° C. (thermal energy ratio: ˜1⅓) and that most enzymes are active in considerably narrower ranges. Consequently, temperature variation has been of limited value in synthetic cell design.

To circumvent this problem, stochastic heating will be used to activate the Polymerase Chain Reaction (PCR) in lipid vesicles in order to amplify their internal DNA copy numbers in a quasi-athermal manner. Once achieved, subsequent divisions of the vesicle will likewise divide the DNA pool among the daughter vesicles, yielding (potentially geometric) growth of a vesicular clone population. In this approach, all of the DNA is made in the progenitor vesicle and becomes more dilute with each division. However, further PCR amplification within the daughter vesicles would re-amplify the DNA supply, enabling a subsequent wave of clone propagation. Previous groups have realized PCR in lipid vesicles. However, these efforts employed conventional PCR which requires cycling the temperature of the system between ˜55° C. and 95° C. Few enzymes (other than DNA Polymerase) can withstand such high temperatures; furthermore, such thermal cycling induces hard convection, among other stresses, that are destructive to vesicles.

The present approach described herein uses stochastic heating to elevate the effective temperature of intra-vesicular DNA while maintaining an essentially constant ambient temperature in the surrounding neutral bath. Because the temperature of the vesicles stays nearly ambient, thermal de-activation of other enzymes and synthetic cell components (ribosomes, mitochondria, molecular machines) that are essential for self-sustaining pseudo-cell populations is inhibited or prevented. This approach may also avoid membrane-damage due to thermal convection.

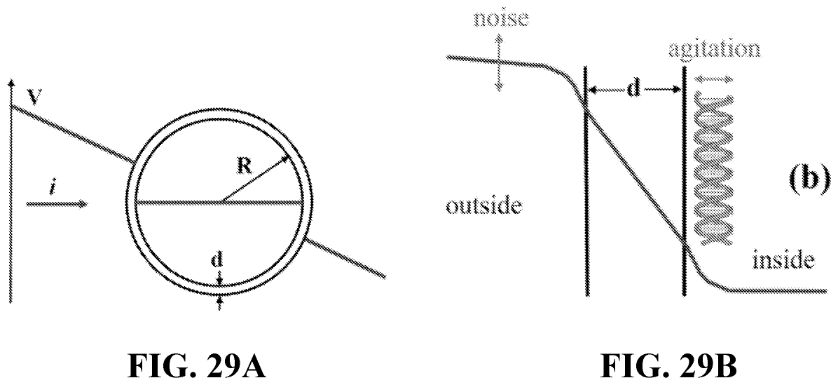

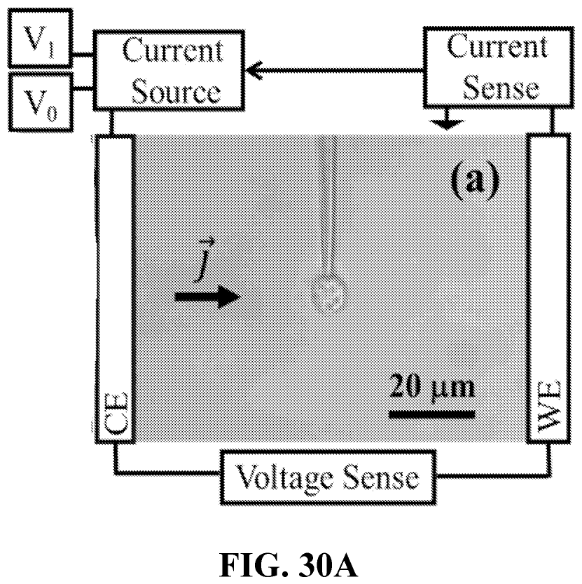

The concept that underlies this radically different approach is depicted in FIG. 29A. When an electrolyte-containing vesicle lies in an external electric field, the presence of the membrane amplifies the field near the membrane. This occurs because the external field E₀ polarizes the electrolytic cytoplasm, creating thin (˜5 nm) screening layers next to the membrane. The potential drop across the cell is now focused across the screening layer and membrane, as shown in FIG. 29B. The field amplification factor in the screening layer is approximately R/d˜10³, where R is the vesicular radius and d is the membrane thickness. Hence, modest external fields become intense in and near the membrane. This field can trap charged species, such as negatively charged DNA molecules, that are in the cytoplasm. The next step is to add a stochastic signal to the external field. This noise-signal randomly oscillates the amplified field in the membrane region of the vesicle and thereby exerts a random force f_{N} on the trapped DNA molecules but not on the surrounding water molecules, which are neutral. The agitated DNA molecules will then have an effective (kinetic) temperature T_{eff} that is greater than the actual temperature of the surrounding bath T:

$${T}_{E f f} ≅ T + k {Γ}^{2} , \qquad ( 2 )$$

where Γ is the amplitude of the noise f_{N}, and k is a constant that converts the noise intensity to temperature. Preliminary data suggest that T_{eff}˜167° C. is attainable in electrolytic solutions. Thus, by stochastically heating a pseudo-cell, it may be possible to use noise to activate the PCR instead of temperature.

A central objective in this example is to vary the amplitude of the external noise signal in order to selectively induce the denaturation, annealing, and elongation steps of the PCR mechanism in lipid vesicles. These three steps occur at ˜95° C., 55° C., and 72° C., respectively, in conventional PCR. Thus, using stochastically-assisted PCR should allow for PCR cycles at lower temperatures, or even room temperature. This would be significant because it would allow PCR to be stimulated in pseudo-cells containing other enzymes and cellular machinery that are essential for self-sustaining populations but that cannot tolerate the high temperatures of PCR.

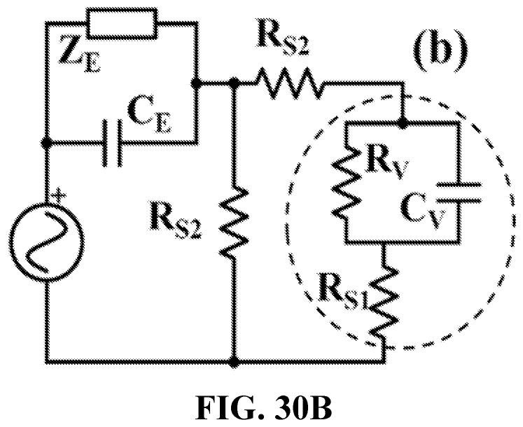

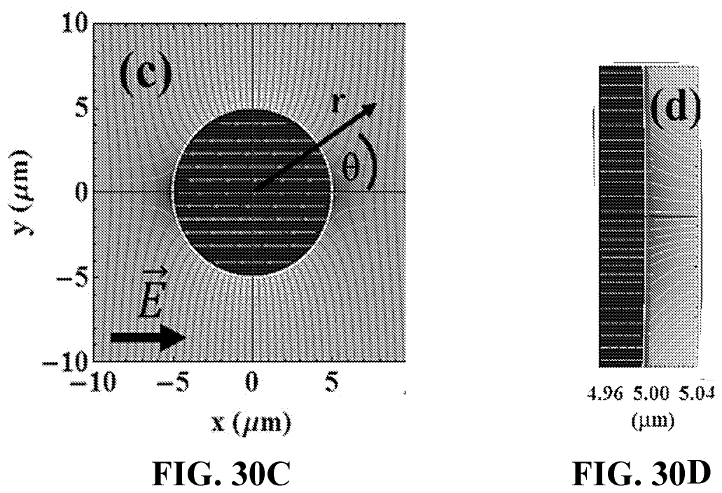

Described herein, therefore, is the design of a sample cell to exert random E-field fluctuations of user-chosen strength on a vesicle in order to stochastically heat charged DNA molecules in the vesicle. In particular, the design of a control circuit that realizes this purpose is described. FIG. 30A depicts a working design for this cell, which is a galvanostatic cell fashioned as two parallel plates with electrolyte+vesicle suspension filling the inter-plate region. A single vesicle in the inter-plate region can be anchored by mild suction to the tip of a pulled pipette. The pipette can be actuated, permitting 3D positioning of the vesicle. The two plates will serve as the counter- and working-electrodes of a circuit that will flow a user-specified current density \vec{j} between the plates. There can be two field components in the sample cell: a steady trapping field \vec{E}₀ and a fluctuating field \vec{E}₁. To properly apply fields \vec{E}₀ and \vec{E}₁ to the vesicle, it is desired that the electrode response to be slow and the vesicular response to be fast. The circuit diagram in FIG. 30B reduces the situation shown in FIG. 30A to an electrode modelled as Z_{E}∥C_{E}, in series with a solution of resistance R_{S2} and the vesicle, denoted by the dashed circle.

To produce the steady trapping field \vec{E}₀, the circuit can generate a steady, spatially uniform current density at positions far from the vesicle. By Ohm's law

$${\vec{E}}_{0} = \frac{{\vec{J}}_{0}}{{σ}_{2}} ,$$

here σ₂ is the electrolyte conductivity; hence, a uniform \vec{j}₀ implies a uniform \vec{E}₀. A vesicle attains an intensified field in its membrane and interfacial regions when exposed to a uniform external field. FIG. 30C is a theoretical picture of a 10 μm vesicle, attained by solving Poisson's equation for the electric potential inside the vesicle, in the membrane (of conductivity σₘ), and outside the vesicle. The potential along the left edge of this plot is approximately +10 μV. The potential drops from left to right. To visualize the intensified field, FIG. 30D shows an enlarged view of the d˜5 nm thick membrane region around θ=0°. The potential drops sharply across this ˜5 nm thickness. Consequently, the radial field is very large in the horizontal poles of the membrane. In fact, when σₘ→0.

$${\vec{E}}_{r} ≅ \frac{1}{2} {E}_{0} ( \frac{{γ}_{V}}{d} ) cos θ \hat{r} , \qquad ( 8 )$$

where E₀ is the magnitude of \vec{E}₀, and the vesicle radius r_{V}=5 μm. Because

$$\frac{{r}_{V}}{d} ≅ {10}^{3} ,$$

the radial field at the poles of the vesicle (around θ=0° and 180°) is very sensitive to the applied field E₀. Furthermore, due to screening of E₀ by the intra-vesicular electrolyte (which this calculation ignored), \vec{E}ᵣ will extend roughly a screening length (˜5 nm) into the interfacial region of the interior electrolyte and, therefore, is useful for trapping charged DNA molecules in this region.

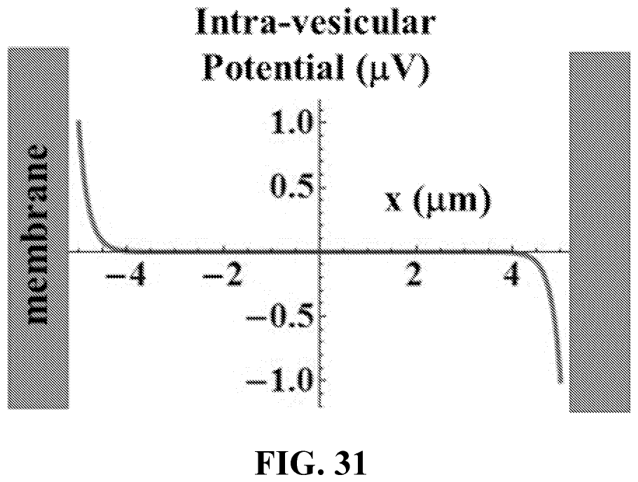

To agitate the trapped DNA molecules, a fluctuating field \vec{E}₁ can be applied by adding stochastic voltage signal V₁ to the control circuit. V₁ can be generated using a broadband function generator (Agilent 81160A). The sample cell response and the vesicular response should be as fast as possible. This is because, within the vesicle, it is desired to have full screening of the field-fluctuation in order to amplify the noise effect in the thin interfacial region of the vesicle. To illustrate this region, the Poisson-Boltzmann equation can be solved for the electric potential in the electrolyte between the L and R sides of a one dimensional, 10 μm diameter vesicle. The potential, plotted in FIG. 31, drops sharply to 0 V near the membrane at +5 μm and −5 μm. The screening length λ_{screen} is exaggerated in this calculation to enable visualization. Via the salt concentration, λ_{screen} can be adjusted to be ˜5 nm in order to easily encompass the DNA molecules. To estimate the vesicular response time, the vesicle can be treated as a spherical resistor R_{V} in parallel with a spherical capacitor

$${C}_{V} = \frac{4 π {r}_{V}^{2} d}{d} {ϵ}_{m} {ϵ}_{0} .$$

The corresponding RC-time constant is approximately:

$${τ}_{V} ∼ ( {R}_{S 1} + {R}_{S 2} ) \frac{4 π {r}_{V}^{2} d}{d} {ϵ}_{m} {ϵ}_{0} .$$

Using a total electrolyte resistance (R_{S1}+R_{S2}) of 300 kΩ (measured), r_{V}˜5 μm, d˜5 nm, and εₘ˜3, τ_{V}˜0.6 μs. This value corresponds to a low-pass frequency f_{3dB}^{V} of ˜0.3 MHz. Through variation of the vesicle size rᵥ(to <500 nm) and solution resistance (to <100 kΩ), this frequency can be varied to ˜80 MHz. The noise bandwidth Δf can be adjusted according to: f_{3dB}^{E}<Δf<f_{3dB}^{V}. As Eqn. (6) indicates, the broader the Δf, the better.

To convert the PCR process from a thermally driven one to a stochastically driven, athermal process one, the following approach will be used: 1) DNA trapping; 2) stochastic denaturation; 3) stochastically-assisted PCR; 4) isothermal and athermal PCR. For each study, many single-vesicle measurements can be used in order to build up solid statistical bases.

0. Vesicle synthesis. Vesicular PCR requires uptake of the necessary PCR components into the interior of the vesicle. Hence, a freeze-drying method can be employed that is known to facilitate large-molecule uptake during the vesicular synthesis. A typical synthesis is as follows. POPC vesicles can be prepared by first producing vesicular dispersions using a standard approach, freeze-drying the dispersion, and then re-hydrating the lyophilized powder with the buffered PCR solution. During rehydration, the aqueous solution carries the large-molecule solutes into the liposome interiors. A vesicle-composition can be employed that has already been used to demonstrate vesicular PCR:POPC:DSPE-PEG₅₀₀:cholesterol (65:5:30). The PCR solution will contain Tris buffer, primer (Sigma, KiCqStart), DNA template, and PCR ReadyMix solution with SYBR Green I dye (Sigma, KCQ500) for double-stranded DNA (ds-DNA) detection. DNase I can be added to the exterior phase to digest ds-DNA and decrease background fluorescence.

1. Interfacial DNA trapping. A single POPC vesicle can be positioned in the sample cell as shown in FIG. 30A. The appropriate E₀-level needed to interfacially trap DNA will be determined as follows. The right-ward directed field is expected to trap DNA along the left edge of the vesicle interior, as the negatively charged DNA molecules will participate in left screening layer. By synthesizing vesicles with large SYBR Green I and DNA concentrations, the occurrence of trapping can be detected by via fluorescence mapping, which will exhibit a bright fringe of fluorescence along the left edge of the vesicle. Further verification can be made by reversing the field in order to shift the fringe to the right side of the vesicle.

2. Stochastic Denaturation: Noise can be applied to single vesicles in order to melt the interfacially trapped ds-DNA molecules. Prior loading of the vesicles with a dye for single-stranded DNA (QuantiFluor ssDNA Dye System) will permit fluorescence-detection of stochastically denatured DNA. Because the radially directed noise field will generally be parallel to a subset of the hydrogen bonds that bind the DNA strands, radial agitation of trapped DNA is expected to efficiently promote denaturation. The field strength E₀ and noise strength Γ can be systematically varied, as well as the DNA template-length, as shorter templates may denature more easily than longer.

3. Stochastically-Assisted PCR: The next step is to realize PCR without thermal cycling to 95° C. After setting the sample cell to 55° C., vesicular DNA can be stochastically denatured (as described above) and then induce annealing simply by turning off the stochastic signal. Annealing occurs spontaneously at 55° C. Thus, a single PCR cycle can be: stochastic denaturation at 55° C.; conventional (55° C.) annealing; and conventional (72°) elongation. Vesicles can be repeatedly run through this cycle until SYBR Green I fluorescence is observed, suggesting amplification. Demonstration of PCR without 95° C. heating will be significant. The next step is to induce elongation stochastically. Ostensibly, a noise-signal of intermediate strength relative to the denaturation-noise level is applied. To focus on this issue, stochastic denaturation can be temporarily omitted and the following PCR cycle can be employed: conventional (95° C.) denaturation; conventional annealing; and stochastic elongation at 55° C. It has been determined that the positively charged ϕ29 residue of DNA polymerase is involved in binding the incoming nucleotide. Without being bound by any theory, it is believed the stochastic field will agitate this charged residue and, thereby, amplify the nucleotide binding rate. Hence, it is believed that elongation can also be stochastically amplified. This protocol can be repeatedly cycled until the SYBR-detection of double-stranded DNA becomes possible.

4. Isothermal and Athermal PCR: Once the methods for stochastic denaturation and stochastic elongation have been established, these capabilities can be combined to enact isothermal 55° C. PCR: stochastic denaturation at 55° C.; conventional (55° C.) annealing; and stochastic elongation at 55° C. The final step is then to perform PCR at room temperature, which will require stochastic annealing. Both the primers and DNA are charged, so it is expected that both will undergo stochastic heating. To identify the optimal conditions, we will prepare 25° C. vesicles that contain primers, ss-DNA, and SYBR Green I. The fluorescence will increase strongly when the primers have annealed to the ss-DNA. We will combine the three processes to realize: stochastic denaturation at 25° C.; stochastic annealing at 25° C.; stochastic elongation at 25° C.

### Example XIV

Stochastic Signals Amplify Electrochemical Current More Effectively than Sinusoidal Signals

In Kramer's theory for chemical barrier crossing, it is the random, fluctuating forces that the solvent exerts on a reactant molecule that drive the reactant over the barrier to the product state. As the temperature sets the fluctuating force-magnitude, these are termed thermally activated reactions. In chemical stochastic heating, the aim is to mimic the random, fluctuating bath-force with an external force in order to amplify the reaction rate. While a stochastic force, such as Gaussian white-noise, seems a natural choice, it is also logical to consider sinusoidally varying external forces. While sinusoidal signals are not random, they share the property of alternating sign with stochastic forces and, hence, may also be effective at rate-amplification. This study compares the chemical rate-enhancement induced by stochastic and sinusoidal signals. The electrochemical deposition of silver is used as a test case. The reaction of interest is: Ag⁺+e⁻→Ag. The focus here is confined to the barrier-limited region of overpotential values, as defined below and will observe the current density of this electrochemical system in order to monitor the rate of this reaction. Below, the experiments show that while sinusoidal forces also increase the reaction rate, they are less effective than Gaussian white noise.

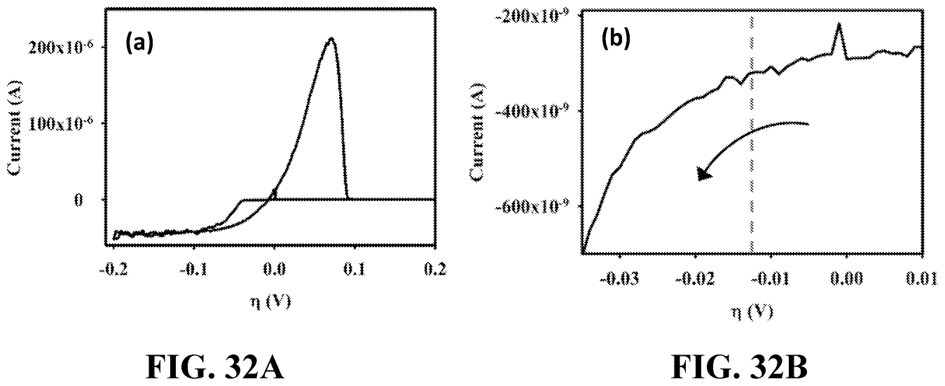

FIG. 32A shows a cyclic voltammogram (CV) collected after multiple (4) oxidation-reduction cycles of a solution containing 5 mM AgNO₃ and 200 mM KNO₃ (the supporting electrolyte). The working electrode was gold. The reference electrode was silver, the counter electrode was silver, and the voltage was scanned at 10 mV/s. This CV agrees well with that reported elsewhere for the same system. Negative current corresponds largely to silver deposition via the Ag⁺+e⁻→Ag process; positive current corresponds to silver dissolution via the Ag→Ag⁺+e⁻ process. FIG. 32B is an expanded view of the barrier-limited region where the current rises exponentially with overpotential (in direction of the arrow). The following studies were performed at an overpotential of −12.5 mV, which provides a driving force for silver electro-deposition.

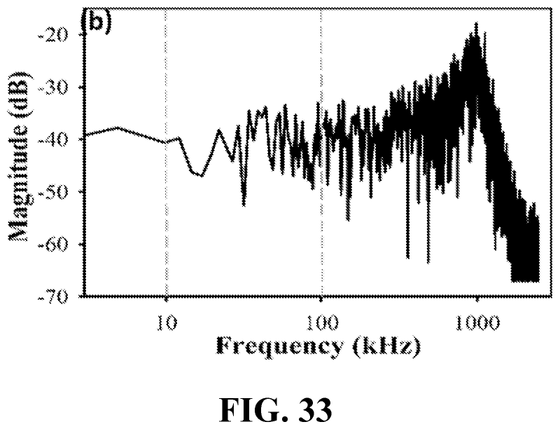

To characterize the rate-amplification capabilities of sinusoidal and stochastic signals, voltage-sweep studies were performed where the current density was recorded and a quartz crystal microbalance (QCM) was used to simultaneously record the deposited mass. Both of these observables permit quantification of electrochemical reaction rates. The working electrode is a gold-coated quartz plate. Essentially, the alternating voltage (stochastic or sinusoidal) is applied between the counter and working-electrodes while controlling its strength, as well as that of the DC overpotential, via a potentiostat and a reference electrode (not shown). By monitoring the resonance frequency of the QCM plate, deposited mass and electrochemical current can be measured simultaneously. The spectrum of the stochastic signal is shown in FIG. 33. Due to potentiostat-limitations, this study is limited to 10 kHz and 100 kHz sine waves (dashed lines in FIG. 33).

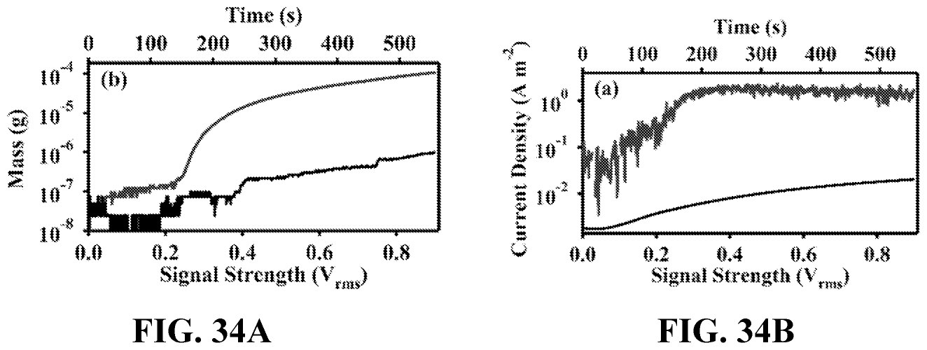

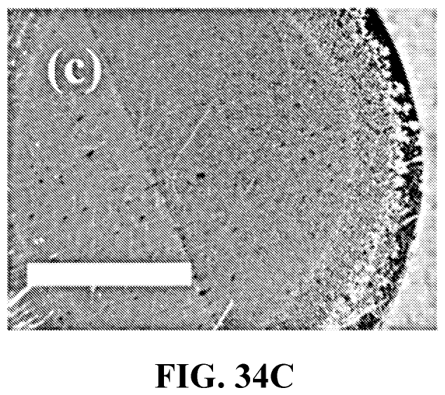

Stochastic rate-amplification and deposited mass enhancement are illustrated in FIGS. 34A-34C. FIG. 34A is a voltage-sweep study that plots the stochastically driven current-density versus the rms-voltage of the stochastic signal across the 0-0.9 V_{RMS} range. The top line is the difference between the total current from a solution containing 5 mM AgNO₃/200 mM KNO₃, and the background-current from a solution having the same ionic strength but no silver: 205 mM KNO₃. The bottom line is the corresponding difference-current density for the DC measurement (i.e. overpotential but no stochastic signal). Visually, the working electrode from the silver-solution had a silvery coating (see FIG. 34C) at the end of the sweep while that from the silver-free solution was uncoated (not shown). Additionally, the QCM indicated no mass deposition from the silver-free solution. To confirm that the stochastic difference-profile corresponds to silver electrodeposition, it has been time-integrated, finding a charge of 98 mC. If due to the Ag⁺+e⁻→Ag process, this charge-value would correspond to an Ag-mass of 110 μg. The deposited mass on the QCM-working electrode is shown in FIG. 34B (top line).

On completion of the voltage-sweep, 107 μg had been deposited. The good agreement between these mass-values

$$\frac{107 μ g}{110 μ g} = 0.97$$

indicates that the Ag⁺+e⁻→Ag process is primarily responsible for the difference-current density and the mass-deposition. Now the degree of amplification that the stochastic signal provides can be assessed. The ratio of the stochastic and DC current densities at the end of the sweeps in FIG. 34A is

$$\frac{1.7 A {m}^{- 2}}{0.0 \; 20 A {m}^{- 2}} = 85 ,$$

indicating a stochastic rate amplification factor of ˜10² for the Ag⁺+e⁻→Ag process. It follows that the stochastic signal also elevates the total deposition-magnitude. The ratio of the stochastic and DC deposition masses in FIG. 34B is

$$\frac{107 μ g}{0.99 μ g} = 108$$

implying a stochastically induced deposition-enhancement of ˜10² for this process. These results establish that the stochastic signal amplifies both the reaction rate and the total deposition-magnitude for the Ag⁺+e⁻→Ag process.

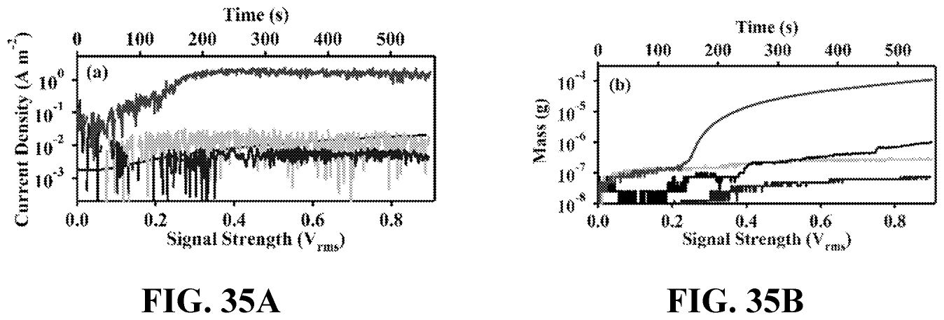

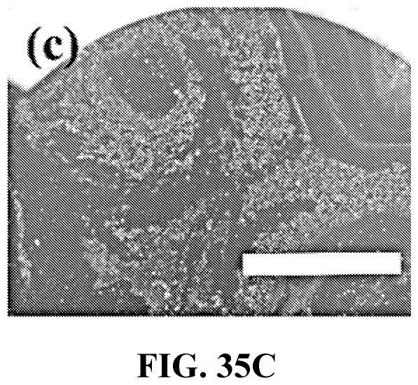

Next, two sinusoidally driven responses were compared. Two profiles in FIGS. 35A and 35B are the stochastic and DC difference-profiles discussed above. The other data-sets are the difference-profiles for 10 kHz and 100 kHz sine waves. The ratio of stochastic-to-sinusoidal current density at, say 0.9′ V_{RMS} is

$$\frac{1.7 A {m}^{- 2}}{0.011 A {m}^{- 2}} = 150$$

at 10 kHz and

$$\frac{1.7 A {m}^{- 2}}{0.0 \; 042 A {m}^{- 2}} = 400$$

at 100 kHz. Across most of the voltage range, the stochastic current density is ˜10²×greater than the sinusoidal current density. FIG. 35B depicts the deposited mass profiles that were collected during these voltage-sweeps. Following an initial period of slow growth (up to ˜150 s or 220 mV_{RMS}), the stochastic mass profile exceeds the sinusoidal mass profiles for all later times. By the end of the sweep, the ratio of stochastic-to-sinusoidal mass is

$$\frac{107 μ g}{0.27 μ g} = 390$$

for the 10 kHz wave and

$$\frac{107 μ g}{0.07 μ g} = 150$$

for the 100 kHz wave. Visually, the sinusoidal deposition-magnitude is weak, as FIG. 35C depicts only a faint silvery coating on the electrode upon completion of the 10 kHz voltage-sweeps. These results demonstrate that relative to sinusoidal waves (with frequencies≤˜100 kHz), stochastic signals significantly amplify the reaction rate and enhance the deposition magnitude for the silver electrodeposition process.

A Gaussian-distributed stochastic signal with a white power spectrum delivers forces of arbitrary magnitude and sequence to a reactant whereas sinusoidal forces are periodic. The results presented in this study strongly suggest that simple periodic forces are not as effective at moving a reactant over an electrochemical barrier as a random force. Furthermore, it is likely that the stochastic signal affects both nucleation and growth phases of the silver deposition process.

### Example XV

Comparison of the translational KE of an Au⁺¹ ion to the rotational KE of an H2O molecule in a field of 10⁹ V m⁻¹

The translational drift velocity of the gold ion in this field is ν_{d}=μₑE=(8×10⁻⁸ C s kg⁻¹)(10⁹ V m⁻¹)=80 m/s, where μₑ is the electrical mobility of the ion. Hence, the quantity of translational kinetic energy that the field adds to the gold ion is

$$Δ {KE}_{trans} = \frac{1}{2} {m}_{A u} {v}_{d}^{2} = 1. x {10}^{- 21} J , \qquad ( 1 a )$$

where the mass of the ion is m_{Au}=3.25×10⁻²⁵ kg.

The rotational drift frequency of a water molecule in this field is

$${ω}_{d} = \frac{{μ}_{d}}{8 π {a}^{3} η} E = ( 260 C s {m}^{- 1} {kg}^{- 1} ) ( {10}^{9} V {m}^{- 1} ) = 2.6 \times {10}^{11} {s}^{- 1} ,$$

where the dipole moment is μ_{d}=1.6 D=5.9×10⁻³⁰ C m, the O—H bond length is a=0.96 Å, and the aqueous viscosity is η=0.001 kg m⁻¹s⁻¹. Hence, the rotational kinetic energy increment that the field adds to the water molecule is

$$Δ {KE}_{rot} = \frac{1}{2} I {ω}_{d}^{2} = 1. x {10}^{- 24} J , \qquad ( 1 b )$$

where the moment of inertia of water is I_{H2O}=2.9×10⁻⁴⁷ kg m².

The ratio

$$\frac{{KE}_{trans}}{{KE}_{rot}} = {10}^{3}$$

shows that the ion's translational degree-of-freedom is considerably more energetic. As a consequence, the field induces a significantly higher effective temperature increase in the ion:

$$Δ {T}_{eff}^{trans} = 2 \frac{{KE}_{trans}}{{k}_{B}} = 148 K \qquad ( 2 a )$$

as compared to the H₂O molecule:

$$Δ {T}_{eff}^{trans} = 2 \frac{{KE}_{rot}}{{k}_{B}} = 0.14 K . \qquad ( 2 b )$$

Note that both of these effects change the kinetic energy of the ion or molecule essentially by providing an external impulse or force. This is quite distinct from the field of electrochemistry, which uses the voltage to lower the barriers for electron transfer between a substrate and an ion/molecule.

### Example XVI

### Demonstrate Growth Via Active Solvent Heating Ionic Liquids (Prophetic)

Several examples above have discussed using a neutral solvent and adding kinetic energy to charged reactants via stochastic heating. Another possibility is to use an ionic solvent that is heated stochastically and then transfers heat to the reactants via collisions. In this case, the real temperature rather than the effective temperature will increase. However, only the solvent at the interface, within approximately a Debye length, will be driven by the electric field and heated. This may enable precise and rapidly changing temperature profiles due to the low thermal mass of the interface region (compared to direct heating of the solvent and/or substrate).

The first testbed will be metal deposition from ionic liquids due to their relatively high thermal stability. In these systems, the solution conductivity increases with the temperature which is expected to increase the deposition rate in addition to thermal fluctuations. Although the example will begin with zinc deposition, this will pave the way for aluminum and/or titanium, which would have applications as coatings for lightweight structures or for extracting rare-earth metals, which have applications within the Critical Materials Institute (a DOE Energy Innovation Hub).

Without being bound by any theory, it is believed that a stochastic voltage signal will elevate the mean kinetic energy of charged molecules in the screening layers of electrode-solution interfaces leading to a temperature increase. The challenge will be to make reliable temperature measurements of a liquid layer that are only a few nanometers thick. Aqueous solutions cannot be used because most molecules are neutral (water), so even in the screening layer a small average temperature rise may occur. For this reason, ionic liquids (ILs) will be used where all particles are charged, leading to a maximal temperature rise (which should be further enhanced by a small 3-5 Å screening layer). Experimentally, both thermal atomic force microscopy and Langmuir film-surface pressure techniques can be used to measure the interfacial temperature. All-atom, molecular dynamics (MD) simulations of the same ILs will be performed under vertical stochastic fields to compute the temperature profile of the IL near the interface. The heating efficiency of stochastic and non-stochastic fields can be compared, including constant, sinusoidal, and sawtooth fields. The significance of this example is that it directly tests the fundamental concept of stochastic heating and provides an estimate of the magnitude of the effect.

The aim of this example is to directly demonstrate a field-dependent temperature rise in regions of the fluid that sustain an electric field (screening layers). To measure the temperature directly at the electrode-solution interface, thin film temperature sensors (such as Omega TFD series) can be sandwiched between electrodes with the top surface of the temperature sensor in contact with the ionic liquid. To ensure that the noise fields do not alter the sensor response, the results will be compared with control experiments in non-polar solvents.

Scanning Thermal Microscopy (SThM) is a scanning microscopy technique using a specialized probe with a small (<50 nm) thermocouple integrated into the tip. To measure temperature profiles perpendicular to the substrate, force curves can be collected as the probe crosses the interface, simultaneously providing the temperature and the cantilever deflection as functions of z-position. Current SThM probes do not work in conductive liquids because they have wires exposed on the back side. For this reason, the temperature profile will be measured at a liquid-liquid interface composed of an ionic liquid (bottom) and a non-polar solvent (top). Appropriate immiscible pairs are i-Butyl-3-methylimidazolium hexafluorophosphate ([BMIm][PF₆]) and octane. This interface will allow the back side of the cantilever to remain in non-conducting solvent while the tip with the thermocouple probes the ionic liquid. Force curves of liquid-liquid interfaces have been demonstrated, and SThM has been performed in non-polar solvents. These will be put together to use SThM to measure temperature at a liquid-liquid interface. Two changes are expected when stochastic noise is applied to the outer electrodes: 1) the temperature profile will develop a peak within the screening layer and then decay across bulk fluid regions in a near linear manner and 2) the slope of the force curves will soften as the viscosity of the ionic liquid decreases. Both effects will indicate heating. Temperature resolution of 0.01° C. and thermal spatial resolution of ˜50 nm are expected based on the probe specifications (App Nano, VertiSense). Because the thermocouple is larger than typical screening lengths, the temperature measurement will average across a temperature gradient and will be a lower bound to the temperature rise. Measurements will be made with a Cypher ES AFM (Asylum Research).

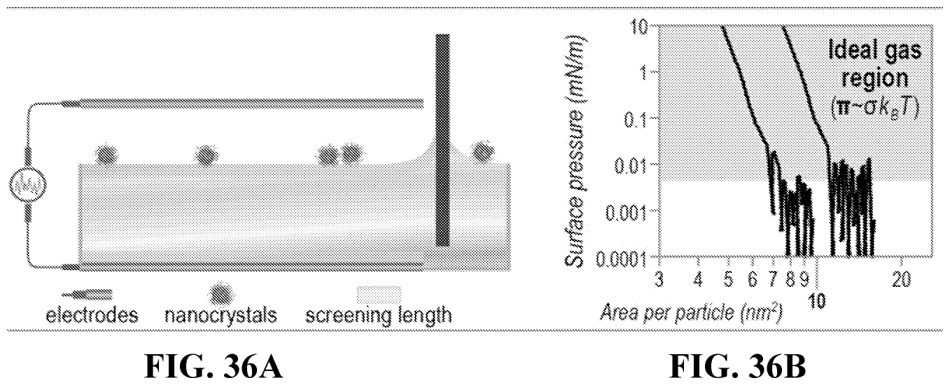

Insoluble particles on air-liquid interfaces form monomolecular films. The surface pressure of these 2D films is easily measured via Langmuir methods, so the strategy is to extract the interfacial temperature from the surface pressure of the film. Using a Langmuir trough, 2D gaseous nanoparticle films will be prepared on an ionic liquid interface [BMIm][PF₆] and a stochastic field will be applied perpendicular to the film while measuring the surface pressure. This arrangement is shown in FIG. 36A. To apply the stochastic field, capacitive plates will be mounted above and below the liquid interface and the stochastic field will be supplied with a function generator. A screening layer in the ionic liquid will form at the air-liquid interface on which the film sits (and at the bottom of the trough). The stochastic field will agitate the molecules in this layer, and the temperature of this layer will be detected. The surface pressure measurements will have ˜10 μN m⁻¹ sensitivity. To extract the temperature, the Carnahan-Starling equation π=k_{B}T(σ+Σₖ₌₂ Bₖ σₖ) will be used to fit the pressure (π)-density (σ) isotherms. The first term is simply the 2D ideal gas law π≅σk_{B}T and describes the initial linear regimes of the pressure-density profiles (see data in FIG. 36B). Using a realistic density of ˜7 particles/nm² and Δπ sensitivity of 10 μN m⁻¹, we estimate the temperature-sensitivity of this approach to be ˜3 K. Because large interfacial temperature changes of 10s of K (or larger) are expected, this sensitivity is sufficient.

Classical all atom molecular dynamics (MD) simulations will be performed to provide atomic level spatial and temporal detail that will illustrate how stochastic fields excite ionic liquid interfaces. Simulations of [BMIm][PF₆] at the interface of a model electrode boundary will be performed using 6×6×12 nm³ rectangular boxes containing approximately 45000 atoms and will be run for a total of 10 μs, (requiring ˜30 days of processing time). The preferred code is Gromacs because it is specifically designed to run efficiently on high performance (either GPU or multi core) platforms, allowing for the simulation of large (10⁵ k atoms) systems for long (μs-ms) times. It is competitive with other common codes (NAMD, CHARIM, AMBER, or LAMMPS). This code allows one to apply external electric fields (both constant and oscillatory). The code will be modified to apply stochastic fields that closely mimic those used experimentally. The rotational and translational diffusion of the ions will be determined, in addition to their local temperature as given by the average kinetic energies. This analysis will yield profiles of the electric field strength and the temperature along the interface-normal. Simulated and measured quantities will be compared.

1. A method of amplifying a reaction rate of one or more ionic reactants within a reaction system comprising the one or more ionic reactants and at least one non-ionic material, the method comprising:
a) generating a stochastic signal with electronic signal generation equipment; and
b) transmitting the stochastic signal to an electrode, the electrode applying the stochastic signal to the reaction system and inducing a stochastic electric field within the reaction system,
wherein the induced stochastic electric field operates to increase the kinetic energy of the one or more ionic reactants to a greater extent than the kinetic energy of the at least one non-ionic material is increased.

2. The method of claim 1, wherein the one or more ionic reactants are present in the reaction system at a concentration of 0.1 mM to 50 mM, and wherein the reaction system has a supporting electrolyte concentration of 1 mM to 1M.

3. The method of claim 1, wherein the stochastic signal has a strength of 10 mV_{RMS} to 10 V_{RMS} and a bandwidth of 100 Hz to 10 MHz.

4. (canceled)

5. The method of claim 1, wherein the reaction system has an initial temperature of 290 K to 305 K.

6. (canceled)

7. The method of claim 1, wherein the electrode surface comprises a material selected from the group consisting of gold, tungsten, platinum, silver, ruthenium, and conducting polymers.

8. The method of claim 1, wherein the induced stochastic electric field operates to increase the kinetic energy of the one or more ionic reactants located within a screening layer having a thickness of about 0.1 nm to about 5 nm.

9. The method of claim 1, wherein the one or more ionic reactants are dispersed or dissolved within the at least one non-ionic material, and wherein the at least one non-ionic material comprises water.

10. (canceled)

11. The method of claim 1, wherein the one or more ionic reactants are present within an ionic liquid, wherein the at least one non-ionic material comprises a neutral gas or liquid forming a separate phase from the ionic liquid, and wherein the electrode is positioned at the interface of the ionic liquid and the separate phase.

12. (canceled)

13. (canceled)

14. A method of amplifying an electrodeposition reaction rate of one or more ionic metal reactants within a reaction system comprising the one or more ionic metal reactants dispersed or dissolved within a solvent, the method comprising:
applying a stochastic electric signal to the reaction system, thereby inducing a stochastic electric field within at least a portion of the reaction system,
wherein the induced stochastic electric field operates to increase the kinetic energy of at least a portion of the one or more ionic metal reactants to above the kinetic energy of the one or more ionic metal reactants at the boiling point of the solvent.

15. The method of claim 14, wherein the one or more ionic metal reactants comprise metal cations of gold (Au), silver (Ag), Copper (Cu), and/or chromium (Cr).

16. (canceled)

17. The method of claim 14, wherein during the applying, the one or more ionic metal reactants reacts to form a metal film on a substrate disposed within the reaction system.

18. A method of amplifying a reaction rate of one or more ionic reactants within a reaction system comprising the one or more ionic reactants dissolved within a solvent and having a system temperature, the method comprising:
applying a stochastic electric signal to the reaction system, thereby inducing a stochastic electric field within at least a portion of the reaction system and increasing the kinetic energy of at least a portion of the one or more ionic reactants without increasing the system temperature above the boiling point of the solvent,
wherein the portion of the one or more ionic reactants having increased kinetic energy reacts to form a precipitate product.

19. The method of claim 18, wherein the applying step comprises applying a first stochastic signal having a first strength to the reaction system to increase a nucleation rate of the precipitate product and applying a second stochastic signal having a second strength to the reaction system to increase a growth reaction rate of the precipitate product.

20. The method of claim 18, wherein the precipitate product comprises calcium carbonate (CaCO₃), magnesium carbonate (MgCO₃), calcium phosphate (CaHPO₄), zinc oxide (ZnO), and/or copper sulfide (CuS).

21. A method of stochastically heating a DNA molecule in a lipid vesicle dispersed within a reaction system, the method comprising:
applying a first electric signal to the reaction system, thereby inducing an electric field adjacent a membrane of the lipid vesicle and trapping the DNA molecule within a screening layer formed by the electric field; and
applying a stochastic electric signal to the reaction system, thereby increasing the kinetic energy of the trapped DNA molecule.

22. The method of claim 21, wherein applying the stochastic electric signal increases the kinetic energy of the DNA molecule to an effective temperature of about 90° C. to about 100° C., thereby melting or denaturing the DNA molecule, wherein the method further comprises, after melting or denaturing the DNA molecule, decreasing the strength of the stochastic electric signal to thereby decrease the kinetic energy of the DNA molecule to an effective temperature of about 50° C. to about 60° C.

23. (canceled)

24. (canceled)

25. Apparatus for amplifying a reaction rate of one or more ionic reactants within a reaction system comprising the one or more ionic reactants and at least one non-ionic material, the apparatus comprising:
electronic signal generation equipment configured to generate a stochastic signal;
a working electrode in electronic communication with the electronic signal generation equipment and disposed within the reaction system, the working electrode configured to apply the stochastic signal to the reaction system and induce a stochastic electric field within the reaction system,
wherein the induced stochastic electric field operates to increase the kinetic energy of the one or more ionic reactants to a greater extent than the kinetic energy of the at least one non-ionic material is increased.

26. The apparatus of claim 25, further comprising an unwired electrode or target substrate, wherein the induced stochastic electric field operates to increase the kinetic energy of the one or more ionic reactants within a screening layer adjacent the unwired electrode or target substrate.

27. The apparatus of claim 25, wherein the electronic signal generation equipment is configured to generate the stochastic signal having a strength of 10 mV_{RMS} to 10 V_{RMS} and a bandwidth of 100 Hz to 10 MHz.

28. (canceled)

29. The apparatus of claim 25, wherein a surface of the working electrode comprises a material selected from the group consisting of gold, tungsten, platinum, silver, ruthenium, and conducting polymers, wherein the one or more ionic reactants are present within an ionic liquid, and wherein the working electrode is positioned at an interface of the ionic liquid and a separate neutral phase.

30. (canceled)
